SEQUENCE LISTING (27 sequences)

SEQ ID NO: 1 - RNA, 20 nt, synthetic construct
  source 1..20: mol_type = other RNA
gtatggcagcaacgtcacga

SEQ ID NO: 2 - RNA, 20 nt, synthetic construct
  source 1..20: mol_type = other RNA
cattgtagtgtccacggtcc

SEQ ID NO: 3 - RNA, 20 nt, synthetic construct
  source 1..20: mol_type = other RNA
gccagggcaaaaccacacag

SEQ ID NO: 4 - RNA, 20 nt, synthetic construct
  source 1..20: mol_type = other RNA
ggctccaaaggacttgtacg

SEQ ID NO: 5 - RNA, 20 nt, synthetic construct
  source 1..20: mol_type = other RNA
gcctgctgtcacttgctacg

SEQ ID NO: 6 - RNA, 20 nt, synthetic construct
  source 1..20: mol_type = other RNA
gcttgcgttagtggtgtact

SEQ ID NO: 7 - RNA, 24 nt, synthetic construct
  source 1..24: mol_type = other RNA
caccgtatggcagcaacgtcacga

SEQ ID NO: 8 - RNA, 24 nt, synthetic construct
  source 1..24: mol_type = other RNA
aaactcgtgacgttgctgccatac

SEQ ID NO: 9 - RNA, 23 nt, synthetic construct
  source 1..23: mol_type = other RNA
caccgaccgtggacactacaatg

SEQ ID NO: 10 - RNA, 23 nt, synthetic construct
  source 1..23: mol_type = other RNA
aaaccattgtagtgtccacggtc

SEQ ID NO: 11 - RNA, 24 nt, synthetic construct
  source 1..24: mol_type = other RNA
caccgccagggcaaaaccacacag

SEQ ID NO: 12 - RNA, 24 nt, synthetic construct
  source 1..24: mol_type = other RNA
aaacctgtgtggttttgccctggc

SEQ ID NO: 13 - RNA, 23 nt, synthetic construct
  source 1..23: mol_type = other RNA
caccgctccaaaggacttgtacg

SEQ ID NO: 14 - RNA, 23 nt, synthetic construct
  source 1..23: mol_type = other RNA
aaaccgtacaagtcctttggagc

SEQ ID NO: 15 - RNA, 24 nt, synthetic construct
  source 1..24: mol_type = other RNA
caccgcctgctgtcacttgctacg

SEQ ID NO: 16 - RNA, 24 nt, synthetic construct
  source 1..24: mol_type = other RNA
aaaccgtagcaagtgacagcaggc

SEQ ID NO: 17 - RNA, 23 nt, synthetic construct
  source 1..23: mol_type = other RNA
caccgtacaccactaacgcaagc

SEQ ID NO: 18 - RNA, 23 nt, synthetic construct
  source 1..23: mol_type = other RNA
aaacgcttgcgttagtggtgtac

SEQ ID NO: 19 - DNA, 18 bp, synthetic construct
  source 1..18: mol_type = other DNA
aatgaacaacaaccgccc

SEQ ID NO: 20 - DNA, 20 bp, synthetic construct
  source 1..20: mol_type = other DNA
cgaacgaatgaacaaacgag

SEQ ID NO: 21 - DNA, 22 bp, synthetic construct
  source 1..22: mol_type = other DNA
caaggaagttactgcactaagg

SEQ ID NO: 22 - DNA, 20 bp, synthetic construct
  source 1..20: mol_type = other DNA
gtgtaactgaaagcaagccc

SEQ ID NO: 23 - DNA, 20 bp, synthetic construct
  source 1..20: mol_type = other DNA
gcagactaacactcactccc

SEQ ID NO: 24 - DNA, 20 bp, synthetic construct
  source 1..20: mol_type = other DNA
tcctatccagccacgaatac

SEQ ID NO: 25 - DNA, 22 bp, synthetic construct
  source 1..22: mol_type = other DNA
cccatcatactgacttctttcc

SEQ ID NO: 26 - DNA, 19 bp, synthetic construct
  source 1..19: mol_type = other DNA
tagaagccaggtgcagtag

SEQ ID NO: 27 - protein, 13 aa, synthetic construct
CGYGRKKRRQRRR

(19) United States
(12) Patent Application Publication
(10) Pub. No.: US 2025/0145975 A1
(43) Pub. Date: May 8, 2025

(54) METHODS FOR IN VIVO DELIVERY AND DETECTION OF CLUSTERED REGULARLY INTERSPACED SHORT PALINDROMIC REPEAT SYSTEMS UTILIZING BACULOVIRUS VECTOR-MAGNETIC NANOPARTICLE COMPLEXES

(71) Applicant: University of Kentucky Research Foundation, Lexington, KY (US)

(72) Inventors: Xiaoyue Yang, Lexington, KY (US); Sheng Tong, Lexington, KY (US)

(21) Appl. No.: 18/941,989

(22) Filed: Nov. 8, 2024

Related U.S. Application Data
(60) Provisional application No. 63/597,227, filed on Nov. 8, 2023.

(51) Int. Cl. C12N 9/22; A61K 9/51; A61K 38/17; A61K 48/00; A61N 2/00; A61P 35/00; C12N 15/11; C12N 15/86

(52) U.S. Cl. CPC C12N 9/22; A61K 9/5115; A61K 38/1774; A61K 48/0008; A61N 2/002; A61P 35/00; C12N 15/111; C12N 15/86; C12N 2310/20; C12N 2710/14043

(57) ABSTRACT

Methods which make use of baculovirus vector (BV)-magnetic nanoparticle (BV-MNP) complexes to facilitate in vivo delivery of clustered regularly interspaced palindromic repeat (CRISPR) systems are provided. BV-MNP complexes carrying a CRISPR nuclease and a guide RNA having homology to an immune checkpoint gene can be administered to a subject to inhibit the immune checkpoint gene. Inhibition of the immune checkpoint gene can promote a desired immune response in the subject. Methods which leverage the contrast provided by MNPs in BV-MNP complexes in combination with magnetic resonance imaging (MRI) to detect in vivo delivery of CRISPR systems are also provided.

### RELATED APPLICATION

This application claims priority to U.S. Patent Application Ser. No. 63/597,227 filed on Nov. 8, 2023, the entire disclosure of which is incorporated herein by reference.

### GOVERNMENT INTEREST

This invention was made with government support under grant numbers: R01EB026893, awarded by the National Institute of Biomedical Imaging and Bioengineering (NIBIB); and R21HL166178, awarded by the National Heart, Lung, and Blood Institute (NHLBI). The government has certain rights in the invention.

### REFERENCE TO AN ELECTRONIC SEQUENCE LISTING

The contents of the electronic sequence listing (Name: 2854 Sequence Listing.xml; Size: 24,935 bytes; Date of Creation: Nov. 8, 2024) is herein incorporated by reference in its entirety.

### TECHNICAL FIELD

The presently disclosed subject matter generally relates to clustered regularly interspaced short palindromic repeat (CRISPR)-based gene editing. In particular, certain embodiments of the presently disclosed subject matter relate to methods which make use of baculoviral vector (BV)-magnetic nanoparticle (MNP) (BV-MNP) complexes to facilitate in vivo delivery of CRISPR systems to inhibit an immune checkpoint gene impeding a desired immune response.

### BACKGROUND

CRISPR/Cas9 technology holds significant potential for cancer therapy due to its ability to edit genomic DNA. It can modify genes crucial to tumor growth, drug resistance, and anti-cancer immunity. However, clinical translation faces challenges due to the lack of a dependable in vivo delivery method. Uncontrolled gene editing in normal tissue can induce genotoxicity. Traditional methods using known viral vectors, lipid nanoparticles, and hydrodynamic injection have limitations such as low delivery efficiency and the risk of viral gene integration.

### SUMMARY

The presently disclosed subject matter meets some or all of the above-identified needs, as will become evident to those of ordinary skill in the art after a study of information provided in this document.

This summary describes several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The presently disclosed subject matter generally includes methods which make use of baculovirus vector (BV)-magnetic nanonparticle (MNP) (BV-MNP) complexes to facilitate in vivo delivery of clustered regularly interspaced palindromic repeat (CRISPR) systems.

Provided are methods for targeted in vivo gene editing. In some embodiments, a method for targeted in vivo gene editing comprises: packaging a CRISPR nuclease and a guide RNA having homology to an immune checkpoint gene impeding an immune response in a subject into a BV; attaching a plurality of MNPs to the BV to form a BV-MNP complex; and administering the BV-MNP complex carrying the CRISPR nuclease and the guide RNA to the subject to thereby inhibit the immune checkpoint gene.

Further provided are methods for prompting an immune response in a subject. In some embodiments, a method for prompting an immune response in a subject comprises: administering a BV-MNP complex carrying a CRISPR nuclease and a guide RNA having homology to an immune checkpoint gene impeding the immune response in the subject, wherein administration of the BV-MNP complex carrying the CRISPR nuclease and the guide RNA inhibits the immune checkpoint gene.

Also provided is a method for inhibiting tumor growth in a subject. In some embodiments, a method for inhibiting tumor growth in a subject comprises: administering a BV-MNP complex carrying a CRISPR nuclease and a guide RNA having homology to an immune checkpoint gene impeding infiltration of at least one of lymphocytes and dendritic cells into the tumor tissue, wherein administration of the BV-MNP complex carrying the CRISPR nuclease and the guide RNA inhibits the immune checkpoint gene.

Methods for detecting in vivo delivery of a CRISPR system are also provided. In some embodiments, a method for detecting in vivo delivery of a CRISPR system comprises: administering a BV-MNP complex carrying a CRISPR nuclease and a guide RNA having homology to a gene to a subject; imaging the subject or a biopsy acquired from the subject with magnetic resonance imaging (MRI) to acquire one or more images; and detecting the presence or absence of the BV-MNP complex based on the presence or absence of a depiction of MNPs in the one or more images. In some embodiments, T₂-weighted MRI is utilized to image the subject or the biopsy.

In some embodiments of the various methods provided, the CRISPR nuclease is CRISPR-associated protein 9 (Cas9). In some embodiments of the various methods provided, the gene or immune checkpoint gene is programmed death-ligand 1 (PDL-1). In some embodiments of the various methods provided, the guide RNA comprises the sequence of SEQ ID NO: 1, SEQ ID NO: 2, SEQ ID NO: 3, SEQ ID NO: 4, SEQ ID NO: 5, or SEQ ID NO: 6.

In some embodiments of the various methods provided, inhibition of the immune checkpoint gene invokes the immune response in the subject which was initially impeded by the immune checkpoint gene. In some embodiments of the various methods provided, the immune response impeded by the immune checkpoint gene is increased infiltration of at least one of lymphocytes and dendritic cells into a tissue of the subject. In some embodiments of the various methods provided, the tissue of the subject is cancerous tissue. In some embodiments of the various methods provided, the tissue is cancerous tumor tissue. In some embodiments of the various methods provided, the subject is diagnosed with or displays symptoms indicative of colon cancer. In some embodiments of the various methods provided, the BV-MNP complex carrying the CRISPR nuclease and the guide RNA is administered in cancerous tissue of the subject. In some embodiments of the various methods provided, inhibition of the immune checkpoint gene results in increased infiltration of lymphocytes including CD3 lymphocytes, CD8 lymphocytes, or a combination thereof into the tissue of the subject.

In some embodiments of the various methods provided, an immune checkpoint inhibitor is administered to the subject in combination with the BV-MNP complex carrying the CRISPR nuclease and the guide RNA. In some embodiments of the various methods provided, the immune checkpoint inhibitor is alpha cytotoxic T-lymphocyte associated protein 4 (αCTLA-4).

In some embodiments of the various methods provided, the method further comprises a step of applying a magnetic field to a target tissue in the subject subsequent to administration of the BV-MNP complex carrying the CRISPR nuclease and the guide RNA, where the target tissue corresponds to an area of the subject where the immune response impeded, at least initially, by the immune checkpoint gene is desired.

In some embodiments of the various methods provided, inhibition of the immune checkpoint gene is characterized by a decrease in expression or activity of a protein encoded by the immune checkpoint gene and/or inhibition of tumor growth in a subject.

Further features and advantages of the present disclosure will become evident to those of ordinary skill in the art after a study of the description, figures, and non-limiting examples in this document.

### BRIEF DESCRIPTION OF DRAWINGS

FIG. 1A are transmission electron microscopy (TEM) images of magnetic nanoparticles (MNP) iron(iii) acetylacetonate (Fe(acac)₃), baculovirus vectors (BV), and BV conjugated to MNP (BV-MNP). Scale bar: 200 μm.

FIG. 1B is a graph showing dynamic light scattering (DLS) analysis of Fe(acac)₃ MNPs.

FIG. 1C is an image showing the results of gel electrophoresis of Fe(acac)₃ MNP without cys-TAT peptide (TAT) (left lane, negatively charged) and Fe(acac)₃ MNP conjugated with TAT (right lane, positively charged).

FIG. 1D is a graph showing enhanced green fluorescent protein (eGFP) expression in MC38 cells infected by BV-MNPs carrying VB1-SpCas9-eGFP (BV-MNP VB1-SpCas9-eGFP) with different MNP amounts, where VB1 corresponds to guide RNA (gRNA) targeting programmed death-ligand 1 (PD-L1). Flow cytometry analysis. Data are shown as the mean±standard deviation (s.d.).

FIG. 1E is a graph showing eGFP expression in MC38 cells decreased over the course of a week after infection with BV carrying eGFP (BV eGFP). Flow cytometry analysis. Data are shown as the mean±s.d.

FIG. 2A are graphs comparing eGFP expression and PD-L1 fluorescent mean in MC38 cells infected by BV eGFP and BV carrying VB1-SpCas9-eGFP (BV VB1-SpCas9-eGFP).

FIG. 2B is a graph showing eGFP expression in MC38 cells after 24 hours (h) post-transduction by BV-eGFP and BV-VB1-SpCas9-eGFP at different multiplicities of infection (MOI).

FIG. 2C is a series of images showing fluorescent imaging of eGFP expression in MC38 cells when transduced by BV in different conditions, including DMEM/F12 medium (F12) and containing 50% adult mouse serum (AMS), and by BV-MNP in F12 medium containing 50% AMS. Blue: nucleus, green: eGFP. Scale bar: 100 μm.

FIG. 2D is a series of graphs showing flow cytometry analysis of eGFP expression in MC38 cells 24 h after BV eGFP transduction and fluorescent mean in MC38 cells 48 h after BV eGFP transduction in different conditions and BV-MNP eGFP transduction (indicated by “MNP-TAT” bar in FIG. 2D) in F12 containing 50% AMS. Data are shown as the mean±s.d., *** represents P<0.001, **** represents P<0.0001. Data analyzed by one-way ANOVA.

FIG. 2E is a series of graphs showing flow cytometry analysis of eGFP expression in MC38 cells 24 h and 48 h after BV VB1-SpCas9-eGFP transduction in different conditions and BV-MNP VB1-SpCas9-eGFP transduction (indicated by “MNP-TAT” bar in FIG. 2E) in F12 containing 50% AMS. Data are shown as the mean±s.d., *** represents P<0.001, **** represents P<0.0001. Data analyzed by one-way ANOVA.

FIG. 2F is a graph showing flow cytometry analysis of PD-L1 knockout percentage in MC38 cells 48 h after BV VB1-SpCas9-eGFP transduction in different conditions and BV-MNP VB1-SpCas9-eGFP transduction (indicated by “MNP-TAT” bar in FIG. 2F) in F12 containing 50% AMS. Data are shown as the mean±s.d., *** represents P<0.001, represents P<0.0001. Data analyzed by one-way ANOVA.

FIG. 3A are graphs showing flow cytometry analysis of PD-L1 knockout efficiency in MC38 cells after 48 h transduction by BV carrying VB1, SpCas9, and near-infrared fluorescent protein (iRFP) (BV VB1-SpCas9-iRFP) and BV carrying only SpCas9 and iRFP (BV SpCas9-iRFP) at different MOIs (20 MOI (upper)) (100 MOI (lower)).

FIG. 3B is a graph showing a comparison of cell growth rate between MC38 cells and 2B12 cells over the course of 72 h at different cell seeding amounts (1000 and 4000) via cell counting kit 8 (CCK8) assay. 2B12 is a clone selected from MC38 cells treated with pX330-VB1 PD-L1 knockout cell clone of MC38.

FIG. 3C are graphs showing the impact of BV carrying VB1, SpCas9, and iRFP (BV VB-Spca9-iRFP) and BV carrying SpCas9 and iRFP without VB1 (BV SpCas9-iRFP) transduction on MC38 cell growth in vitro at different MOIs at 24 h (upper) and 48 h (lower) post-transduction via CCK8 assay.

FIG. 3D is a graph showing cytotoxicity via CCK8 assay of splenocytes towards MC38 cells transduced by BV VB1-SpCas9-iRFP or BV SpCas9-iRFP and control uninfected MC38 cells at different splenocyte-to-MC38 cell ratios.

FIG. 3E is a series of graphs comparing gene expression (Interferon-gamma (IFN-γ), Granzyme B (GranB), Interleukin-2 (IL-2), and Interleukin-10 (IL-10)) in splenocytes co-cultured with MC38 cells transduced by BV VB1-SpCas9-iRFP (as indicated by “VB1” in FIG. 3E), BV SpCas9-iRFP (as indicated by “SpCas9” in FIG. 3E), or lipopolysaccharide (LPS) after 24 h by real-time PCR (RT-PCR). Data are shown as the mean±s.d., ** represents P<0.01, *** represents P<0.001, **** represents P<0.0001. Data analyzed by one-way ANOVA.

FIG. 3F is a graph showing the cytotoxicity of splenocytes towards MC38 cells (splenocyte:MC38 cells=5:1) transduced by BV VB1-SpCas9-iRFP (as indicated by “VB1” in FIG. 3F), MC38 cells transduced by BV SpCas9-iRFP (as indicated by “Spcas9” in FIG. 3F), and control uninfected MC38 cells after 24 h by CCk8 assay. Data are shown as the mean±s.d., ** represents P<0.01, *** represents P<0.001. Data analyzed by one-way ANOVA.

FIG. 4A is a volcano map of gene expression difference (x-axis corresponding to fold change and y-axis corresponding to p-value) in MC38 cells and BV VB1-SpCas9-iRFP transduced MC38 cells. Data points above horizontal line considered to be statistically relevant. Data points left of first (left-most) vertical line indicate gene down regulation in cells, data points right of second (right-most) vertical line indicate gene upregulation in cells, data points between first and second vertical line indicate no significant difference in gene regulation in cells. nCounter tumor signaling 360 panel analysis of the whole RNA isolated from MC38 and BV VB1-SpCas9 transfected MC38 cells revealed that among the 506 genes analyzed, 26 genes were significantly downregulated (p-Adj <0.05, fold change ≤−1.5), and 81 genes were significantly upregulated (p-Adj <0.05, fold change ≥1.5).

FIG. 4B is a heat map showing a comparison of the signaling pathway expression in control MC38 cells and BV VB1-SpCas9-iRFP transduced MC38 cells using data from FIG. 4A. Data analyzed by nSolver analysis software. Blue coloration corresponds to downregulated pathways. Orange coloration corresponds to upregulated pathways. Black coloration corresponds to pathways without significant change. Brackets indicate relation between indicated pathways. Pathways indicated from top to bottom are: Autophagy; JAK-STAT Signaling; Cell Adhesion & Motility; Remodeling & Metastasis; PDGF Signaling; Glucose Metabolism; HIF1 Signaling; Cell Cycle; DNA Damage Repair; PI3K-Akt Signaling; FGFR Signaling; Apoptosis; ERBB2 Signaling; EGFR Signaling; T-cell Exhaustion; Estrogen Signaling; MET signaling; VEGF Signaling; Hippo Signaling; Wnt Signaling; TGF-beta Signaling; Interleukin Signaling; Hedgehog; Nrf2 & Oxidative Stess; EMT; Antigen Presentation; Senescence; Inflammation; Androgen Signaling; p53 Signaling; Transcriptional Regulation; MAPK Signaling; Myc; NF-kB Signaling; Interferon Response; Chemokine Signaling; Immortality & Sternness; and mTOR Signaling.

FIG. 5A is a perspective view of a magnetic bed including a magnetic device for applying a magnetic field to act on MNPs.

FIG. 5B is an image of the magnetic bed of FIG. 5A in use.

FIG. 5C is a schematic diagram of a tumor positioned above a quadrupole provided by one set of cylindrical magnets provided in the magnetic bed of FIG. 5A.

FIG. 5D is an image showing simulated magnetic force acting on the surface of the tumor of FIG. 5C. Simulation generated using MATLAB program developed in-house.

FIG. 5E is an image showing simulated magnetic force throughout the mass of the tumor of FIG. 5C. Simulation generated using MATLAB program developed in-house.

FIG. 6A is a series of unprocessed and processed, T₂ overlay magnetic resonance imaging (MRI) images of tumors in BV VB1-SpCas9-iRFP (upper) and BV-MNP VB1-SpCas9-iRFP (lower) transduced C57BLC mice following MC38 cancer cell infusion treatment.

FIG. 6B is an image of cryosection of a tumor of FIG. 6A transduced with BV-MNP VB1-SpCas9-iRFP. Clusters of MNPs (black dots) are visible near the peripheral of the tumor. The arrow points to the direction of the magnetic force.

FIG. 6C is a series of T₂ images (left) and processed, T₂ overlay (map) (right) MRI images of tumor samples in C57BLC mice transduced with BV-MNP VB1-SpCas9-iRFP, MNP, BV VB1-SpCas9-iRFP, and PBS.

FIG. 7A is a series of immunofluorescent images acquired by an inverted fluorescence microscope (Nikon Ti2) of representative tumor sections of C75BLC mice implanted with murine MC38 cancer cells and subsequently treated with PBS, MNP, BV VB1-SpCas9-iRFP, αPD-L1 antibody, and BV-MNP VB1-SpCas9-iRFP stained for CD3 and CD8.

FIG. 7B is a graph showing in vivo efficacy of PBS treatment on tumor volume in C75BLC mice implanted with murine MC38 cancer cells (n=8). Tumor volumes were measured every three days with vernier caliper and calculated with known formula (V=0.5×(L×W²)).

FIG. 7C is a graph showing in vivo efficacy of MNP treatment on tumor volume in C75BLC mice implanted with murine MC38 cancer cells (n=8). Tumor volumes were measured every three days with vernier caliper and calculated with known formula (V=0.5×(L×W²)).

FIG. 7D is a graph showing in vivo efficacy of BV VB1-SpCas9-iRFP treatment on tumor volume of C75BLC mice implanted with murine MC38 cancer cells (n=8). Tumor volumes were measured every three days with vernier caliper and calculated with known formula (V=0.5×(L×W²)).

FIG. 7E is a graph showing in vivo efficacy of BV-MNP VB1-SpCas9-iRFP treatment on tumor volume in C75BLC mice implanted with murine MC38 cancer cells (n=8). Tumor volumes were measured every three days with vernier caliper and calculated with known formula (V=0.5×(L×W²)).

FIG. 7F is a graph showing in vivo efficacy of αPD-L1 antibody treatment on tumor volume in C75BLC mice implanted with murine MC38 cancer cells (n=8). Tumor volumes were measured every three days with vernier caliper and calculated with known formula (V=0.5×(L×W²)).

FIG. 7G is a graph using the data from FIGS. 7B-7F and showing in vivo efficacy of BV VB1-SpCas9-iRFP (as indicated by “BV” in FIG. 7G) and BV-MNP VB1-SpCas9-iRFP (as indicated by “BV-MNP” in FIG. 7G) treatment as compared to PBS, MNP and αPD-L1 antibody treatment on tumor volume (n=8 for each group) in C75BLC mice implanted with murine MC38 cancer cells. Data are shown as the mean±s.e.m., ns represents not significant, * represents P<0.05, **** represents P<0.0001. Analyzed by two-way ANOVA.

FIG. 7H is a graph showing a survival curve of C57BLC mice implanted with murine MC38 cancer cells treated with BV VB1-SpCas9-iRFP (as indicate by “BV” in FIG. 7H) and BV-MNP VB1-SpCas9-iRFP (as indicated by “BV-MNP” in FIG. 7H), PBS, MNP, or αPD-L1 antibody (n=8 for each group). Data are shown as the mean±s.e.m., ns represents not significant, * represents P<0.05, **** represents P<0.0001. Analyzed by Log-rank (Mantel-Cox) test.

FIG. 7I is a graph showing body weight of C57BLC mice implanted with murine MC38 cancer cells treated with BV VB1-SpCas9 (as indicate by “BV” in FIG. 7I) and BV-MNP VB1-SpCas9 (as indicated by “BV-MNP” in FIG. 7I), PBS, MNP, or αPD-L1 antibody (n=8 for each group). Data are shown as the mean±s.e.m. Analyzed by two-way ANOVA.

FIG. 7J are images showing Prussian blue staining of representative tumor sections of C75BLC mice implanted with murine MC38 cancer cells and subsequently treated with intratumor injection with PBS (right) or BV-MNP VB1-SpCas9-iRFP (left). Scale bar: 250 μm.

FIG. 7K is a series of graphs illustrating BV-MNP VB1-SpCas9-iRFP transduction as compared to PBS treatment in various cellular components present in tumors of C75BLC mice implanted with murine MC38 cancer cells.

FIG. 7L is a graph using the data from FIG. 7K and indicating the percentage of the various cellular components present in tumors of C57BLC mice implanted with murine MC38 cancer cells and treated with PBS or transduced with BV-MNP VB1-SpCas9-iRFP.

FIG. 7M is a series of immunofluorescent images of representative tumor sections of C75BLC mice implanted with murine MC38 cancer cells and subsequently treated with PBS, MNP, BV VB1-SpCas9-iRFP, αPD-L1 antibody, and BV-MNP VB1-SpCas9-iRFP stained for CD11C and CD86.

FIG. 8A is a graph showing in vivo efficacy of PBS treatment on tumor volume in C75BLC mice implanted with murine MC38 cancer cells (n=8).

FIG. 8B is a graph showing in vivo efficacy of αCTLA-4 antibody treatment on tumor volume in C75BLC mice implanted with murine MC38 cancer cells (n=10).

FIG. 8C is a graph showing in vivo efficacy of αPD-L1 antibody and αCTLA-4 antibody treatment on tumor volume in C75BLC mice implanted with murine MC38 cancer cells (n=8).

FIG. 8D is a graph showing in vivo efficacy of BV-MNP VB1-SpCas9-iRfP and αCTLA-4 antibody treatment on tumor volume in C75BLC mice implanted with murine MC38 cancer cells (n=8).

FIG. 8E is a graph showing a survival curve of C57BLC mice implanted with murine MC38 cancer cells treated with PBS, αCTLA-4 antibody, αPD-L1 antibody and αCTLA-4 antibody, or BV-MNP VB1-SpCas9-iRFP and αPD-L1 antibody (as indicated by “BV-MNP+αCTLA-4” in FIG. 8E (n=8-10 for each group). Data are shown as the mean±s.e.m., ns represents not significant, ** represents P<0.01, *** represents P<0.001. Analyzed by Log-rank (Mantel-Cox) test.

FIG. 8F is a graph showing body weight of C57BLC mice implanted with murine MC38 cancer cells treated with PBS, αCTLA-4 antibody, αPD-L1 antibody and αCTLA-4 antibody, or BV-MNP VB1-SpCas9-iRFP and αPD-L1 antibody (as indicated by “BV-MNP+αCTLA-4” in FIG. 8F) (n=8-10 for each group). Data are shown as the mean±s.e.m. Analyzed by two-way ANOVA.

FIG. 9A is a series of graphs comparing gene expression (cluster of differentiation 86 (CD86), Interferon-gamma (IFN-γ), Tumor necrosis factor alpha (TNF-α), Interleukin-2 (IL-2), Interleukin 10 (IL-10), CD40, Interferon alpha-1 (IFN-α1), Interferon beta-1 (IFN-β1), arginase 1 (ARG1)) in bone marrow derived dendritic cells (BMDC) co-cultured with MC38 cells transduced by BV VB1-SpCas9-iRFP (as indicated by “VB1” in FIG. 9A), BV SpCas9-iRFP (as indicated by “SpCas9” in FIG. 9A), or LPS after 24 h by real-time PCR (RT-PCR). Data are shown as the mean±s.d., ** represents P<0.01, *** represents P<0.001, * represents P<0.0001. Data analyzed by one-way ANOVA.

FIG. 9B is a graph showing the cytotoxicity of BMDCs towards MC38 cells (BMDC:MC38 cells=2:1) transduced by BV VB1-SpCas9-iRFP (as indicated by “VB1” in FIG. 9B), MC38 cells transduced by BV SpCas9-iRFP (as indicated by “Spcas9” in FIG. 9B), and control uninfected MC38 cells after 24 h by CCk8 assay. Data are shown as the mean±s.d., ** represents P<0.01, ns represents not significant. Data analyzed by one-way ANOVA.

FIG. 10 is an image showing the results of a T7E1 assay for sgRNA sequences VB1-VB6 and corresponding exon controls.

### BRIEF DESCRIPTION OF THE SEQUENCE LISTING

SEQ ID NO: 1 is a nucleic acid sequence of an embodiment of a guide RNA sequence in accordance with the presently disclosed subject matter (VB1).

SEQ ID NO: 2 is a nucleic acid sequence of an embodiment of a guide RNA sequence in accordance with the presently disclosed subject matter (VB2)

SEQ ID NO: 3 is a nucleic acid sequence of an embodiment of a guide RNA sequence in accordance with the presently disclosed subject matter (VB3).

SEQ ID NO: 4 is a nucleic acid sequence of an embodiment of a guide RNA sequence in accordance with the presently disclosed subject matter (VB4).

SEQ ID NO: 5 is a nucleic acid sequence of an embodiment of a guide RNA sequence in accordance with the presently disclosed subject matter (VB5).

SEQ ID NO: 6 is a nucleic acid sequence of an embodiment of a guide RNA sequence in accordance with the presently disclosed subject matter (VB6).

SEQ ID NO: 7 is a nucleic acid sequence of an embodiment of a forward oligonucleotide for a guide RNA sequence (VB1) in accordance with the presently disclosed subject matter.

SEQ ID NO: 8 is a nucleic acid sequence of an embodiment of a reverse oligonucleotide for a guide RNA sequence (VB1) in accordance with the presently disclosed subject matter.

SEQ ID NO: 9 is a nucleic acid sequence of an embodiment of a forward oligonucleotide for a guide RNA sequence (VB2) in accordance with the presently disclosed subject matter.

SEQ ID NO: 10 is a nucleic acid sequence of an embodiment of a reverse oligonucleotide for a guide RNA sequence (VB2) in accordance with the presently disclosed subject matter.

SEQ ID NO: 11 is a nucleic acid sequence of an embodiment of a forward oligonucleotide for a guide RNA sequence (VB3) in accordance with the presently disclosed subject matter.

SEQ ID NO: 12 is a nucleic acid sequence of an embodiment of a reverse oligonucleotide for a guide RNA sequence (VB3) in accordance with the presently disclosed subject matter.

SEQ ID NO: 13 is a nucleic acid sequence of an embodiment of a forward oligonucleotide for a guide RNA sequence (VB4) in accordance with the presently disclosed subject matter.

SEQ ID NO: 14 is a nucleic acid sequence of an embodiment of a reverse oligonucleotide for a guide RNA sequence (VB4) in accordance with the presently disclosed subject matter.

SEQ ID NO: 15 is a nucleic acid sequence of an embodiment of a forward oligonucleotide for a guide RNA sequence (VB5) in accordance with the presently disclosed subject matter.

SEQ ID NO 16 is a nucleic acid sequence of an embodiment of a reverse oligonucleotide for a guide RNA sequence (VB5) in accordance with the presently disclosed subject matter.

SEQ ID NO: 17 is a nucleic acid sequence of an embodiment of a forward oligonucleotide for a guide RNA sequence (VB6) in accordance with the presently disclosed subject matter.

SEQ ID NO: 18 is a nucleic acid sequence of an embodiment of a reverse oligonucleotide for a guide RNA sequence (VB6) in accordance with the presently disclosed subject matter.

SEQ ID NO: 19 is a nucleic acid sequence of an embodiment of a forward primer for polymerase chain reaction (PCR) for guide RNAs (VB1, VB4, VB6) in accordance with the presently disclosed subject matter.

SEQ ID NO: 20 is a nucleic acid sequence of an embodiment of a reverse primer for PCR for guide RNAs (VB1, VB4, VB6) in accordance with the presently disclosed subject matter.

SEQ ID NO: 21 is a nucleic acid sequence of an embodiment of a forward primer for PCR for a guide RNA (VB2) in accordance with the presently disclosed subject matter.

SEQ ID NO: 22 is a nucleic acid sequence of an embodiment of a reverse primer for PCR for a guide RNA (VB2) in accordance with the presently disclosed subject matter.

SEQ ID NO: 23 is a nucleic acid sequence of an embodiment of a forward primer for PCR for a guide RNA (VB3) in accordance with the presently disclosed subject matter.

SEQ ID NO: 24 is a nucleic acid sequence of an embodiment of a reverse primer for PCR for a guide RNA (VB3) in accordance with the presently disclosed subject matter.

SEQ ID NO: 25 is a nucleic acid sequence of an embodiment of a forward primer for PCR for a guide RNA (VB5) in accordance with the presently disclosed subject matter.

SEQ ID NO 26 is a nucleic acid sequence of an embodiment of a reverse primer for PCR for a guide RNA (VB3) in accordance with the presently disclosed subject matter.

SEQ ID NO: 27 is an amino acid sequence of an embodiment of a peptide in accordance with the presently disclosed subject matter (TAT).

### DESCRIPTION OF EXEMPLARY EMBODIMENTS

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it is understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently disclosed subject matter, representative methods, devices, and materials are described herein.

The present application can “comprise” (open ended), “consist of” (closed ended), or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about,” when referring to a value or to an amount of mass, weight, time, volume, size, concentration, or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

It is appreciated that where reference is made herein to magnetic nanoparticles (MNPs) being attached or conjugated to a baculovirus vector (BV) to form a BV-MNP complex (BV-MNP) to facilitate delivery of a clustered regularly interspaced short palindromic repeat (CRISPR) system to in vitro or in vivo cells that the MNPs of the BV-MNP will typically have a cell-penetrating peptide (CPP), such as TAT peptide, attached thereto unless indicated otherwise or context precludes.

As used herein, the term “guide RNA” or “gRNA” includes crRNA, tracrRNA and their combination (sgRNA). A crRNA sequence contains the target RNA sequence to locate the correct section of DNA, which binds to e.g., a CAS9 nuclease-recruiting sequence “trans-activating crRNA” (tracrRNA) to form a single guide RNA (sgRNA). The term “guide RNA” or gRNA” also includes crRNA, tracrRNA, and sgRNA with chemical modifications, with additional RNA sequences for tagging and binding to other proteins.

As used herein, the term “Cas9” or “CRISPR-associated protein 9” is a nuclease that functions in a CRISPR system. It is the most commonly used “CRISPR nuclease.” The term Cas9 includes any member of the Cas9 family of genes/proteins or synthetic variants or fusion proteins thereof that function in a CRISPR system, as well as deactivated Cas9 (dCas9). Examples include Streptococcus pyogenes Cas9 (SpCAS9) and Staphylococcus aureus (SaCas9). The term “Cas9” also includes modified Cas9 or dCAS9 proteins with amino acid sequence deletion, insertion and/or mutation.

Other CRISPR nucleases may be utilized in various embodiments of the presently disclosed subject matter. For example, nuclease Cpf1 discovered in the CRISPR/Cpf1 system of the bacterium Francisella novicida, and CjCAS9 from Campylobacter jejuni. Other CRISPR nucleases include Cas8a, Cas5, Cas8b, Cas8c, Cas10d, Cse1, Cse2, Csy1, Csy2, Csy3, GSU0054, Cas10, Csm2, Cmr5, Cas10 or Csx11, Csx10, Csf1, Csn2, Cas4, Cpf1, C2cI, C2c3, and C2c2.

As used herein, “gene editing” includes both functional changes to a protein's activity as well as changes in gene regulation.

It is appreciated that the “MNPs” referred to herein are magnetically responsive particle.

Although the CRISPR systems delivered by BV-MNP complexes are generally referred to herein as including a CRISPR nuclease (e.g., Cas9) and guide RNA (e.g., VB1 (corresponding to SEQ ID NO: 1)), it is appreciated that alternative embodiments in which the CRISPR system carried by a BV-MNP complex further includes a donor template are also contemplated.

A “magnetic device” refers to any device which provides or can create a magnetic field and is thus inclusive of permanent magnets, electromagnets, electropermanent magnets, and assemblies which include one or more permanent magnets, electromagnets, and/or electropermanent magnets. Examples of rare earth magnets suitable for use with the presently disclosed subject matter include, but are not limited to, neodymium rare earth magnets, samarium-cobalt rare earth magnets, Nd2Fe14B, SmCo5, Sm(Co, Fe, Cu, Zr)7, YCO5, or any combination thereof.

Particular types of rare earth magnets may also be selected as desired according to the conditions to which the rare earth magnets may be exposed. For example, any of the following factors may be considered in selecting a type of rare earth magnet: remanence (Br) (which measures the strength of the magnetic field), coercivity (Hci) (the material's resistance to becoming demagnetized), energy product (BHmax) (the density of magnetic energy), and the Curie temperature (Tc). Generally, rare earth magnets have higher remanence, much higher coercivity and energy product than other types of magnets. Where high magnetic anisotropy is desired, YCO₅ may be suitable for use.

The presently disclosed subject matter is based, at least in part, on the discovery that magnetic nanoparticles (MNPs) can be attached to a baculovirus vectors (BV) to form BV-MNP complexes that: (i) are resistive to the effects of the complement system; and (ii) can be utilized to effectively deliver CRISPR systems for inhibiting a target immune checkpoint gene to promote a desired immune response in a subject.

Accordingly, in one aspect, the presently disclosed subject matter includes methods for targeted in vivo gene editing that include: packaging a CRISPR nuclease and a guide RNA targeting (having homology to) an immune checkpoint gene that is impeding a desired immune response in a subject into a BV; attaching a plurality of MNPs to the BV to form a BV-MNP complex (BV-MNP); and administering the BV-MNP complex carrying the CRISPR nuclease and the guide RNA to a subject to thereby inhibit the immune checkpoint gene. By inhibiting the immune checkpoint gene, the desired immune response is thus promoted via administration of the BV-MNP complex carrying the CRISPR nuclease and the guide RNA.

In some embodiments, the CRISPR nuclease is Cas9. In some embodiments, Cas9 is SpCas9. In addition to the MNPs specifically described herein, suitable MNPs which may be utilized in various embodiments of the method also include those disclosed in U.S. Pat. No. 10,286,073, which is incorporated herein by reference in its entirety.

The MNPs of the BV-MNP complex (BV-MNP) can vary in size. In some embodiments, the MNPs of the BV-MNP complex have an average diameter size ranging from about 1 nm to about 200 nm. In some embodiments the MNPs of the BV-MNP complex have an average diameter size that is less than 200 nm. In some embodiments, the MNPs of the BV-MNP complex have an average diameter size ranging from about 1 nm to about 100 nm. In some embodiments, the MNPs of the BV-MNP complex have an average diameter size that is less than 100 nm. In some embodiments, the MNPs of the BV-MNP complex have an average diameter size ranging from about 5 nm to about 50 nm. Without wishing to be bound by any particular theory, it is believed that MNPs with a diameter size ranging from about 5 nm to 50 nm provide particularly good magnetic drag while still facilitating sufficient vector binding. In some embodiments, the BV may have a length of about 250 nm and a diameter of about 30 nm.

In some embodiments, the immune checkpoint gene corresponds to a gene which encodes for a protein that inhibits lymphocyte, such as T lymphocyte (T cell), or dendritic cell infiltration. Accordingly, in some embodiments, the immune response impeded by the immune checkpoint gene is increased infiltration of lymphocytes and/or dendritic cells into a tissue of a subject, and administration of the BV-MNP complex carrying the CRISPR nuclease and the guide RNA results increased infiltration of lymphocytes and/or dendritic cells into the tissue of the subject. In some embodiments, administration of the BV-MNP complex carrying the CRISPR nuclease and the guide RNA results in increased infiltration of CD3 lymphocytes, CD8 lymphocytes, or a combination thereof into the tissue of the subject. In some embodiments, the tissue may correspond to cancerous tissue, such as cancerous tumor tissue. Thus, in some embodiments, the BV-MNP complex carrying the CRISPR nuclease and the guide RNA may be administered to a subject suffering from cancer as a means of providing or supplementing immunotherapy to the subject. For instance, in some embodiments, the subject may be diagnosed with and/or exhibit signs consistent with colon cancer.

In some embodiments, the immune checkpoint gene is programmed death-ligand 1 (PD-L1), also known as cluster of differentiation 274 (CD274), which encodes for the PD-L1 protein. As further discussed below, guide RNAs comprising the sequence of SEQ ID NO: 1, SEQ ID NO: 2, SEQ ID NO: 3, SEQ ID NO: 4, SEQ ID NO: 5, and SEQ ID NO: 6 have been found to facilitate targeting of the PD-L1. Thus, in some embodiments, the guide RNA comprises SEQ ID NO: 1, SEQ ID NO: 2, SEQ ID NO: 3, SEQ ID NO: 4, SEQ ID NO: 5, and SEQ ID NO: 6.

In some embodiments, the method may further include applying a magnetic field to enhance transduction of the BV-MNP complex carrying the CRISPR nuclease and the guide RNA in a target area of the subject. For instance, in some embodiments, subsequent to administration of the BV-MNP complex carrying the CRISPR nuclease and the guide RNA, a magnetic field may be applied to a target tissue of the subject corresponding to an area of the subject where the immune response promoted by inhibition of the immune checkpoint gene is desired to occur. In some embodiments, administration of the BV-MNP complex carrying the CRISPR nuclease and the guide RNA may comprise depositing (e.g., via injection) the BV-MNP complex carrying the CRISPR nuclease and guide RNA directly into the target tissue of the tissue. In some embodiments, the target tissue may be a cancerous tissue, such as tumor, present in the subject. Generation of the magnetic field can be provided by a magnetic device.

It has further been surprisingly discovered that administering the BV-MNP complex carrying the CRISPR nuclease and guide RNA in combination with an immune checkpoint inhibitor can improve treatment outcomes in certain subjects. Thus, in another aspect, the presently disclosed subject matter is directed to a combination therapy in which the BV-MNP complex carrying the CRISPR nuclease and the guide RNA is administered in combination with an immune checkpoint inhibitor. In some embodiments, the immune checkpoint inhibitor is alpha cytotoxic T-lymphocyte associated protein 4 (αCTLA-4). In some embodiments, the immune checkpoint inhibitor is administered to the subject concurrently with the BV-MNP complex carrying the CRISPR nuclease and the guide RNA. In some embodiments, one of the immune checkpoint inhibitor and the BV-MNP complex carrying the CRISPR nuclease and the guide RNA is administered to the subject at a first time and the other of the immune checkpoint inhibitor and the BV-MNP complex carrying the CRISPR nuclease and the guide RNA is administered to the subject at a second time that is different than the first time.

In another aspect, the presently disclosed subject matter includes methods for promoting an immune response in a subject that include: administering a BV-MNP complex carrying a CRISPR nuclease and a guide RNA to the subject, where the guide RNA targets (i.e., has homology to) an immune checkpoint gene impeding a desired immune response in the subject, wherein administration of the BV-MNP complex carrying the CRISPR nuclease and the guide RNA inhibits the immune checkpoint gene. The various CRISPR nucleases, guide RNAs, immune checkpoint genes, and the immune response impeded by the immune checkpoint gene/promoted by inhibition of the immune checkpoint gene described above in relation to the method for targeted in vivo gene editing can be utilized in various embodiments of the method for promoting an immune response in a subject. Administration of the BV-MNP complex carrying the CRISPR nuclease and the guide RNA can be provided to promote immune response at a target tissue of the subject, which, in some embodiments, may be cancerous tissue, such as a tumor.

In some embodiments, the method for promoting an immune response in a subject further includes applying a magnetic field to the target tissue subsequent to administration of the BV-MNP carrying the CRISPR nuclease and the guide RNA to enhance transduction in an area of the subject to which the magnetic field is applied. Generation of the magnetic field can be provided by a magnetic device.

In some embodiments, the method for promoting an immune response in a subject includes administering the BV-MNP complex carrying the CRISPR nuclease and the guide RNA in combination with an immune checkpoint inhibitor. In some embodiments, the immune checkpoint inhibitor is alpha cytotoxic T-lymphocyte associated protein 4 (αCTLA-4). In some embodiments, the immune checkpoint inhibitor is administered to the subject concurrently with the BV-MNP complex carrying the CRISPR nuclease and the guide RNA. In some embodiments, one of the immune checkpoint inhibitor and the BV-MNP complex carrying the CRISPR nuclease and the guide RNA is administered to the subject at a first time, and the other of the immune checkpoint inhibitor and BV-MNP complex carrying the CRISPR nuclease and the guide RNA is administered to the subject at a second time that is different than the first time.

In another aspect, the presently disclosed subject matter includes methods for inhibiting tumor growth in a subject that includes: administering a BV-MNP complex carrying a CRISPR nuclease and a guide RNA targeting (i.e., having homology to) an immune checkpoint gene to tumor tissue in the subject, wherein administration of the BV-MNP complex carrying the CRISPR nuclease and the guide RNA inhibits the immune checkpoint gene. The various CRISPR nucleases, guide RNAs, immune checkpoint genes, and the immune response impeded by the immune checkpoint gene/promoted by inhibition of the immune checkpoint gene described above in relation to the methods above can be utilized in various embodiments of the method for inhibiting tumor growth in a subject. In some embodiments, the method includes applying a magnetic field to the tumor targeted for treatment and/or administering the BV-MNP complex carrying the CRISPR nuclease and gRNA in combination with an immune checkpoint inhibitor, such as αCTLA-4, as described above.

Inhibition of the immune checkpoint gene and/or promotion of the desired immune response in the various methods disclosed herein can, in some embodiments, be characterized, at least in part, by a decrease in the expression or activity of the protein encoded by the immune checkpoint gene and/or inhibition of tumor growth in a subject using various methods/techniques disclosed herein or readily known to those of skill in the art. Decreases in the expression or activity of the protein encoded by the immune checkpoint may, in some embodiments, be determined by comparing protein expression or activity at different points in time (e.g., pre-administration and post-administration of the BV-MNP carrying the CRISPR nuclease and guide RNA) and/or via comparison to protein expression or activity exhibited in a control subject or relative to predetermined expression or activity values indicative of protein expression or activity in a control subject. In various embodiments of the presently disclosed subject matter, inhibition of the immune checkpoint gene targeted by the guide RNA may be assessed utilizing methods selected from the group consisting of ELISA, Luminex, FACs, Western blot, dot blot, immunoprecipitation, immunohistochemistry, immunocytochemistry, immunofluorescence, immunodetection methods, optical spectroscopy, radioimmunoassay, mass spectrometry, HPLC, qPCR, RT-qPCR, multiplex qPCR, SAGE, RNA-seq, microarray analysis, FISH, MassARRAY technique, and combinations thereof. Inhibition of tumor growth may, in some embodiments, be determined by measuring the size of a tumor in a subject at different time points and/or via comparison to a control subject.

In another aspect, the presently disclosed subject matter includes methods for detecting in vivo delivery of a CRISPR system that include: administering a BV-MNP complex carrying a CRISPR nuclease and a guide RNA targeting (i.e., having homology to) a gene to a subject; imaging the subject or a biopsy acquired from the subject with magnetic resonance imaging (MRI) to acquire one or more images; and detecting the presence or absence of the BV-MNP complex based on the presence or absence of a depiction of MNPs in the one or more images. Presence and non-presence of a depiction of MNPs in the one or more images signifies delivery and non-delivery, respectively, of the CRISPR system by the BV-MNP complex. Biopsies can be acquired and prepped for analysis utilizing known techniques. The various CRISPR nucleases and guide RNAs described in relation to the methods above can be utilized in various embodiments of the method for detecting in vivo delivery of the CRISPR system. In some embodiments, the gene targeted by the guide RNA is an immune checkpoint gene consistent with that referred to above. In some embodiments, imaging of the subject or the biopsy may include T₂-weighted MRI imaging. In some embodiments, the method includes applying a magnetic field to a target tissue of the subject subsequent to the administration of the BV-MNP carrying the CRISPR nuclease and the guide RNA and prior to imaging of the subject or the biopsy acquired from the subject. In some embodiments, the biopsy may be a biopsy of cancerous tissue, such as tumor tissue. In some embodiments, the cancerous tissue may be attributed to colon cancer in the subject.

As reflected in the discussion above, the BV-MNP complexes including the CRISPR nucleases and guide RNAs disclosed herein may, alone or in combination with an immune checkpoint inhibitor, such as α-CTLA-4, act and be administered as, or a component of, a therapeutic composition to treat a subject in need thereof. Such subjects may include cancer patients. In some instances the subject may be diagnosed or exhibit symptoms of colon cancer.

Except where context precludes, suitable methods for administering a therapeutic composition in accordance with the presently-disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, dermally (e.g., topical application), intratracheal installation, surgical implantation, transdermal delivery, local injection, intranasal delivery, and hyper-velocity injection/bombardment. In some embodiments of the therapeutic methods described herein, the therapeutic compositions are administered intravenously to treat a disease or disorder.

Regardless of the route of administration, the therapeutic agents used in accordance with the presently-disclosed subject matter are typically administered in an amount effective to achieve the desired response. As such, the term “effective amount” is used herein to refer to an amount of the therapeutic composition sufficient to produce a measurable biological response. Actual dosage levels of active ingredients in a therapeutic composition used in accordance with the presently-disclosed subject matter can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, New Jersey; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Florida; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pennsylvania; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

The present methods can be used on a wide variety of subjects. Indeed, the term “subject” as used herein is not particularly limited. The term “subject” is inclusive of vertebrates, such as mammals, and the term “subject” can include human and veterinary subjects. Thus, the subject of the herein disclosed methods can be a human, non-human primate, horse, pig, rabbit, dog, sheep, goat, cow, cat, guinea pig, rodent, or the like. The term does not denote a particular age or sex. Thus, adult and newborn subjects, as well as fetuses, whether male or female, are intended to be covered.

The presently disclosed subject matter is further illustrated by the following specific but non-limiting examples. Further, the following examples may include compilations of data that are representative of data gathered at various times during the course of development and experimentation related to the present invention.

### EXAMPLES

### Introduction

CRISPR technology presents a promising avenue for addressing disease-causing genetic mutations. Recently, several CRISPR therapies have gained FDA approval for the treatment of β-hemoglobinopathies, and ongoing exploration is underway for curing a diverse array of genetic diseases. CRISPR employs various Cas nucleases and guide RNAs (gRNAs) to locate and modify specific genomic loci, rendering it a versatile tool for gene deletion, insertion, modification, or regulation. In contrast to small-molecule drugs, CRISPR has distinct advantages, including rational design, swift turnaround time, and multiplexed gene editing capabilities. Notably, it achieves target protein modification by editing corresponding genomic loci, resulting in complete and permanent changes in edited cells. These unique features of CRISPR, combined with recent advancements in clinical sequencing techniques, open the door to personalized and multiplexed immunomodulation in the TME through gene editing approaches.

However, in vivo therapeutic applications of CRISPR gene editing involve substantial challenges. gRNA can bind to DNA sequences with base mismatches, and Cas nucleases can tolerate base mismatches and DNA and RNA bulges, leading to off-target activities. Such off-target activities may induce tumorigenic mutations and other adverse effects. In in vivo therapeutic gene editing, the systemic dissemination of the CRISPR system and its long-term presence present risks of genotoxicity. Existing gene-editing techniques fall short in controlling CRISPR activities spatially and temporally. For instance, viral vectors are commonly utilized in vivo gene editing due to their high transduction efficiency and ability to transduce non-dividing cells. Nevertheless, viral vectors tend to distribute throughout the body following systemic or local injection. Viral vectors of mammalian origin can induce long-term transgene expression by integrating into the host genome or exhibiting stable extrachromosomal expression. Due to these concerns, therapeutic gene editing currently undergoing clinical trials is performed in vivo in limited anatomical regions, such as the subretinal space, where the spread of viral vectors is restricted, or ex vivo using isolated hematopoietic stem/progenitor cells.

Baculoviral vectors (BV) have a high transduction efficiency and low cytotoxicity in various cell lines, primary cells, and stem cells, making them robust vehicles for gene delivery. Unlike non-viral vectors, BV can efficiently transduce non-dividing cells, including resting stem cells, by directly releasing its genomic materials into the cell nuclei. Importantly, BV cannot replicate in mammalian cells due to its insect origin. BV-mediated transgene expression is transient, lasting only a few days, and any BV that escapes into the systemic circulation can be neutralized by the complement system. These properties minimize off-target activity of CRISPR. In comparison to adeno-associated virus (AAV), commonly used as a carrier for CRISPR, BV has a larger DNA packing capacity (>38 kb in BV vs. 5 kb in AAV). This allows the entire gene circuit, including Cas9 nucleases, gRNAs, genetic regulatory elements, and reporter genes, to be packaged into the same BV. Such consolidation thus enhances gene regulation efficacy, simplifies the system, and reduces production costs.

As disclosed, for example in U.S. Pat. No. 10,286,073, which is incorporated herein by reference in its entirety, previous research has shown that MNPs conjugated with a cell penetrating peptide, TAT (CGGSYGRKKRRQRRR) (MNP-TAT) can bind to BV. The complex of MNP-TAT and BV can then be pulled down using a magnet plate, and the pulldown efficiency could be reduced by heparin, a naturally occurring polymer with a strong negative charge. Similar observations were made when MNPs were conjugated with poly(arginine), indicating that MNP-TAT binds to BV through electrostatic interactions.

Previous studies have also shown that BV can be inactivated by the complement system in the serum. BV inactivation involves C3-mediated surface binding and membrane attacking. In this regard, we previously found that BV-induced transgene expression in murine hepatoma Hepa 1-6 cells could be abolished by the culture medium containing 50% of adult mouse serum (AMS). To examine the effect of MNPs on BV, BV expressing luciferase was mixed with MNPs and incubated with Hepa 1-6 cells under various conditions. In the serum-free medium, incubation on a magnetic plate for 30 minutes was enough to induce high luciferase expression, indicating that MNP could enhance BV transduction under a magnetic field (MF). Hepa 1-6 cells showed negligible BV uptake and BV-mediated transgene expression in the medium containing 50% of AMS. However, the application of MF significantly enhanced the endocytosis of MNP-BV and restored BV transduction in the same medium. The combination of MNPs and MF is required for protecting BV from serum inactivation. Magnetic activation of BV is facilitated by the increased local accumulation, accelerated endocytosis, and enhanced viral nuclear entry due to actin polymerization induced by the magnetic force.

We previously evaluated BV-MNP transduction in nude mice carrying s.c. tumors. MNP-BV carrying a luciferase gene was administrated via intratumoral infusion, and a block magnet was placed on the tumor for one hour following infusion. We found high luciferase activity within the tumor tissue, while all vital organs showed no signs of transduction. Both in vitro and in vivo studies consistently demonstrate that the combination of BV-MNP and MF can induce localized BV transduction through kinetic competition with the complement system. This phenomenon occurs as the MF enhances the margination of MNP-BV in the bloodstream, promotes interaction with the cell membrane, and ultimately facilitates viral entry into the cells. Lastly, NGS sequencing proved that gene editing was detectable only in the tumor tissue.

The studies underlying the current examples focus on the use of BVs in the development of immunotherapies which disrupt target immune checkpoints that impede a desired immune response. In particular, certain aspects of the studies underlying the current examples focus on BV-mediated transduction of CRISPR-associated nucleases and gRNAs in vivo that induce an antitumor immune response by inhibiting specific immune checkpoint genes in cancer cells. In this regard, BV-mediated delivery of CRISPR-associated nucleases and gRNAs targeting specific immune checkpoint genes in vivo can prompt double-stranded breaks (DSBs) in the targeted genes. In the absence of a donor template, DSBs will be repaired via error-prone non-homologous end joining (NHEJ) pathway, leading to mutational insertions and deletions (indes) of short sequences at the repair sites. Indels occurring in coding exons can cause changes in individual amino acids or frameshift mutations, resulting in nonfunctional proteins or splicing errors which can inhibit reactivation of the disrupted gene checkpoint and thus facilitate a desire, such as anti-tumor, immune response.

### Materials and Methods

### Preparation of Magnetic Nanoparticles (MNPs)

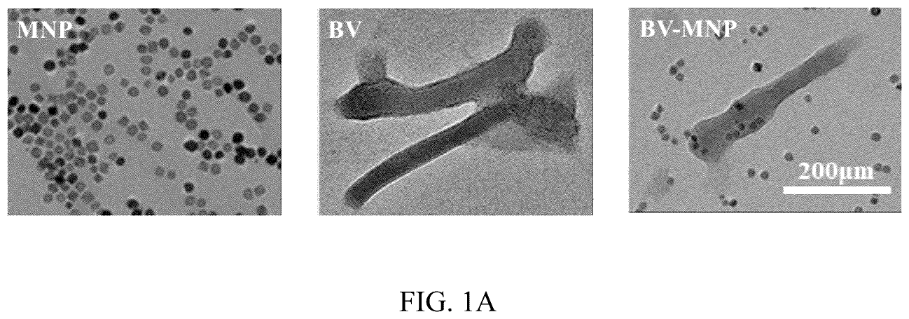

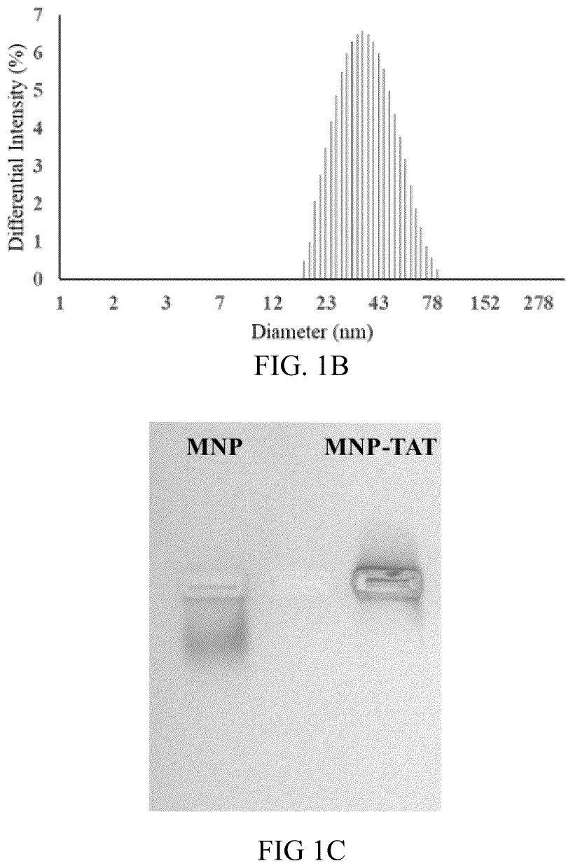

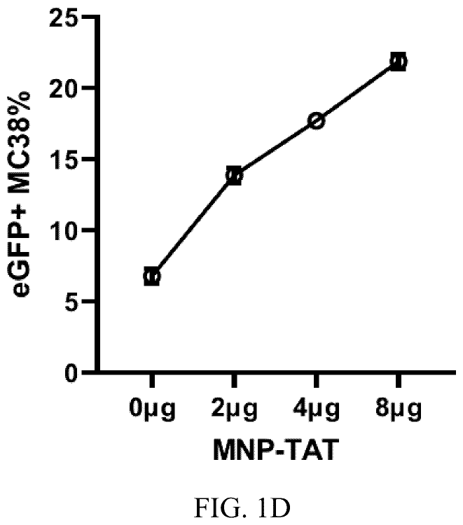

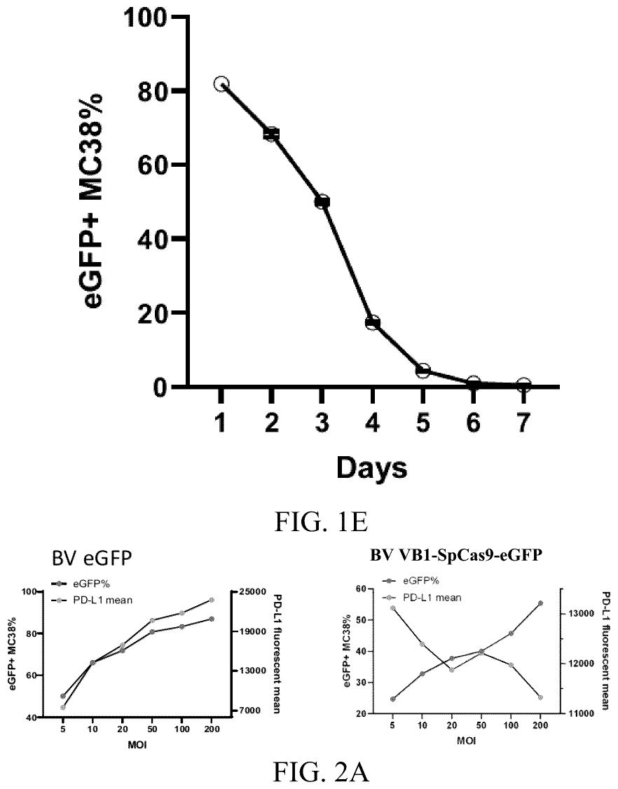

MNPs were synthesized in a manner consistent with known protocols.^{25, 29, 30} In brief, nanocrystals were synthesized through thermodecomposition of iron(iii) acetylacetonate (Fe(acac)₃) in a mixture of benzyl ether, oleic acid, and oleylamine. Water-dispersible MNPs were generated by coating the nanocrystals with DSPE-PEG2000-methoxy and DSPE-PEG2000-maleimide in a molar ratio of 20:1 using a dual solvent exchange method. MNPs were conjugated to a positively charged peptide which can be attached to the surface of BVs (FIGS. 1A and 1C). In this regard, freshly coated MNPs were mixed with cys-TAT (CGYGRKKRRQRRR (SEQ ID NO: 27)) peptides in 0.2× phosphate-buffered saline (PBS) and incubated overnight. Unconjugated peptides were removed by ultracentrifugation. The physical properties of MNPs were characterized using transmitted electron microscopy (TEM) (FIG. 1A) and dynamic light scattering (DLS) analysis (FIG. 1B). Gel electrophoresis was utilized to verify conjugation of TAT peptides to the MNPs (FIG. 1C).

### Baculoviral Vector (BV) Constructions

BV constructs referred to in the drawings and studies disclosed herein were constructed by the Bac-to-Bac® Baculovirus Expression System (Thermo Fisher Scientific) according to distributor protocols. BV derived from Autographa californica multicapsid nucleopolyhedroviurs. Recombinant bacmids were transduced into Sf9 insect cells using Cellfectin® II reagent. BV of passage 3 was utilized in the studies disclosed herein.

BV was conjugated to MNPs to form BV-MNP complexes through electrostatic interaction between the viral envelope and the TAT peptides on the surface of MNPs (FIG. 1A). MNPs were found to have an average size less than 200 μm (FIG. 1A). Streptococcus pyogenes CRISPR-associated protein 9 (SpCas9) was utilized as CRISPR-associated endonuclease.

It is appreciated that although reference is sometimes made to BV-mediated transduction involving the use of BV or BV-MNP to transport a CRISPR system which includes green fluorescent protein (eGFP) near-infrared fluorescent protein (iRFP) (in addition to the guide RNA (gRNA) and CRISPR-associated endonuclease) that such proteins are utilized for tagging purposes to more easily detect transduction and do not assist with treatment efficiency. Accordingly, the studies disclosed herein involving BV-mediated transduction involving BV or BV-MNP to transport system including eGFP or iRFP could similarly be carried out without eGFP or iRFP in instances where imaging or detection facilitated by eGFP or iRFP is not needed.

Guide RNAs (gRNAs) Targeting Programmed Death-Ligand 1 (PD-L1)

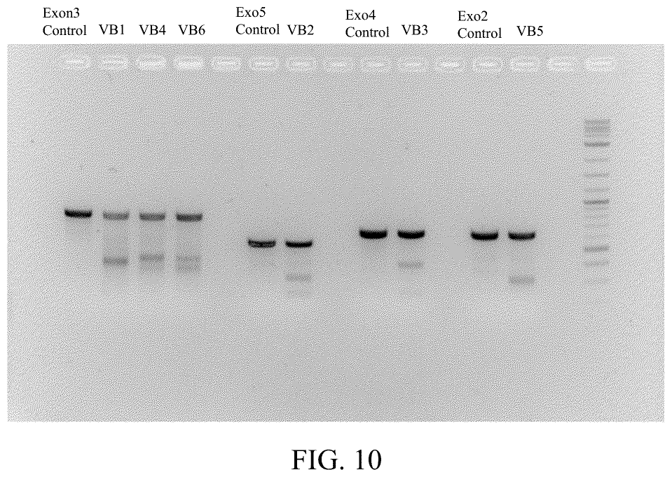

Guide RNAs targeting PD-L1 were designed using a bioinformatics tool (available at https://www.vbc-score.org/) based on a Vienna Bioactivty CRISPR (VBC) scoring method and screened in vitro in cultured mouse MC38 colon cancer cells using pX330 plasmids purchased from Addgene and corresponding exon controls (Table 1 and FIG. 10).²⁴ gRNA targeting exon 3 of mouse PD-L1 and referred to herein as “VB1”, was selected according to editing efficiency (Table 1). TIDE analysis showed that approximately 90% of indels induced in PD-L1 gene led to frameshift mutations (data not shown). gRNAs were synthesized by Eurofins Genomics. Sequences for the gRNAs targeting PD-L1 and corresponding oligonucleotides are provided in Tables 2 and 3, respectively.

TABLE 1
Cutting Efficiency of Guide RNAs Targeting PD-L1.

| | Sample | Cutting Efficiency (%) | R² |
|---|---|---|---|
| 1 | Exon3 Control | Uncut | |
| 2 | pX330-VB1 | 24.4% | 0.96 |
| 3 | pX330-VB4 | 20.5% | 0.99 |
| 4 | pX330-VB6 | 14.5% | 0.98 |
| 5 | Exon5 Control | Uncut | |
| 6 | pX330-VB2 | 10.7% | 0.94 |
| 7 | Exon4 Control | Uncut | |
| 8 | pX330-VB3 | 6.56% | 0.97 |
| 9 | Exon2 control | Uncut | |
| 10 | pX330-VB5 | 8.80% | 0.91 |

TABLE 2
sgRNA Sequences of Guide RNAs Targeting PD-L1.

| Name | sgRNA sequence |
|---|---|
| VB1 | GTATGGCAGCAACGTCACGA (SEQ ID NO: 1) |
| VB2 | CATTGTAGTGTCCACGGTCC (SEQ ID NO: 2) |
| VB3 | GCCAGGGCAAAACCACACAG (SEQ ID NO: 3) |
| VB4 | GGCTCCAAAGGACTTGTACG (SEQ ID NO: 4) |
| VB5 | GCCTGCTGTCACTTGCTACG (SEQ ID NO: 5) |
| VB6 | GCTTGCGTTAGTGGTGTACT (SEQ ID NO: 6) |

TABLE 3
Oligonucleotides for sgRNAs Targeting PD-L1.

| Name | Oligonucleotide |
|---|---|
| VB1 | Forward: CACCGTATGGCAGCAACGTCACGA |
| | (SEQ ID NO: 7) |
| | Reverse: AAACTCGTGACGTTGCTGCCATAC |
| | (SEQ ID NO: 8) |
| VB2 | Forward: CACCGACCGTGGACACTACAATG |
| | (SEQ ID NO: 9) |
| | Reverse: AAACCATTGTAGTGTCCACGGTC |
| | (SEQ ID NO: 10) |
| VB3 | Forward: CACCGCCAGGGCAAAACCACACAG |
| | (SEQ ID NO: 11) |
| | Reverse: AAACCTGTGTGGTTTTGCCCTGGC |
| | (SEQ ID NO: 12) |
| VB4 | Forward: CACCGCTCCAAAGGACTTGTACG |
| | (SEQ ID NO: 13) |
| | Reverse: AAACCGTACAAGTCCTTTGGAGC |
| | (SEQ ID NO: 14) |
| VB5 | Forward: CACCGCCTGCTGTCACTTGCTACG |
| | (SEQ ID NO: 15) |
| | Reverse: AAACCGTAGCAAGTGACAGCAGGC |
| | (SEQ ID NO: 16) |
| VB6 | Forward: CACCGTACACCACTAACGCAAGC |
| | (SEQ ID NO: 17) |
| | Reverse: AAACGCTTGCGTTAGTGGTGTAC |
| | (SEQ ID NO: 18) |

Polymerase chain reaction (PCR) primers for T7E1 assay are provided in Table 4. Exon controls were prepared by PCR with same pair of primers of each gRNA and the whole genome of MC38 as the DNA template.

TABLE 4
Primers for T7E1 assay.

| Name | Primer |
|---|---|
| VB1, VB4, | Forward: |
| VB6 | AAT GAA CAA CAA CCG CCC |
| | (SEQ ID NO: 19) |
| | Reverse: |
| | CGA ACG AAT GAA CAA ACG AG |
| | (SEQ ID NO: 20) |
| VB2 | Forward: |
| | CAA GGA AGT TAC TGC ACT AAG G |
| | (SEQ ID NO: 21) |
| | Reverse: |
| | GTG TAA CTG AAA GCA AGC CC |
| | (SEQ ID NO: 22) |
| VB3 | Forward: |
| | GCA GAC TAA CAC TCA CTC CC |
| | (SEQ ID NO: 23) |
| | Reverse: |
| | TCC TAT CCA GCC ACG AAT AC |
| | (SEQ ID NO: 24) |
| VB5 | Forward: |
| | CCC ATC ATA CTG ACT TCT TTC C |
| | (SEQ ID NO: 25) |
| | Reverse: |
| | TAG AAG CCA GGT GCA GTA G |
| | (SEQ ID NO: 26) |

### BV-Mediated Transduction In Vitro

To assess in vitro BV-mediated transduction, various tests on MC38 cells purchased from ATCC, a model for colorectal cancer, were performed.

To assess the effect of different amounts (0 μg, 2 μg, 4 μg, 8 μg) of MNP-TAT on eGFP expression in MC38 cells transduced by BV, BV carrying VB1-SpCas9-eGFP (BV VB1-SpCas9-eGFP) was used. 4×10⁴ MC38 cells/well were cultured in 24-well plate 24 hours before BV transduction. BV VB1-SpCas9-eGFP was used at multiplicity of infection (MOI) 100 and mixed with different doses of MNP-TAT at room temperature for 20 minutes. BV-MNP was added into the medium and cultured with MC38 cells for two hours, then the medium containing BV-MNP was discarded, and the cells were cultured in fresh medium for 18-24 hours before flow cytometry to detect eGFP expression in MC38 cells (FIG. 1D).

To assess the duration of eGFP expression in MC38 cells post-BV-mediated transduction, BV carrying eGFP (BV eGFP) was used. 4×10⁴ MC38 cells/well were cultured in 24-well plate 24 hours before BV transduction. BV eGFP was used at MOI 50 and co-cultured with MC38 cells for 24 hours. MC38 cells were collected and analyzed with flow cytometry to detect eGFP expression in MC38 cells (FIG. 1E).

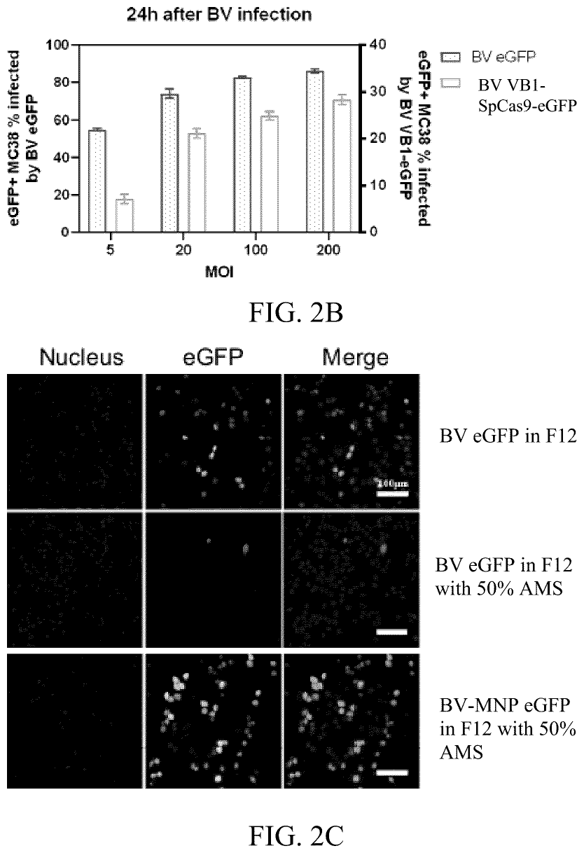

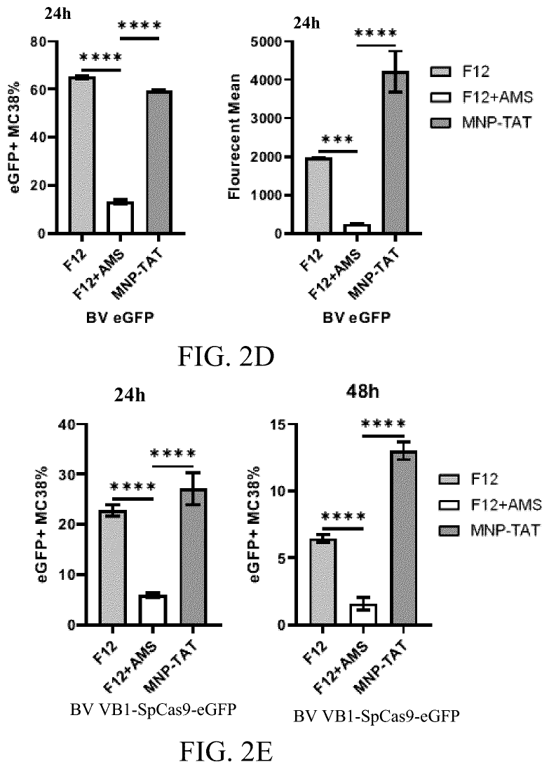

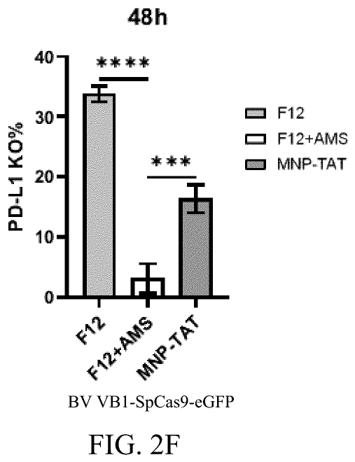

To assess BV-mediated eGFP expression in MC38 cells at different MOIs, MC83 cells were transduced with BV eGFP and BV VB1-SpCas9-eGFP. 4×10⁴ MC38 cells were seeded in 24-well plate each well. 24 hours later, BV eGFP and BV VB1-SpCas9-eGFP were added into the fresh medium and cultured with MC38 cells, respectively. 24 hours after BV eGFP transduction, MC38 cells were collected, stained with anti-mouse PD-L1 antibody, washed with PBS and analyzed with flow cytometry to determine the eGFP expression in MC38 cells and PD-L1 protein on MC38 cell membrane. 48 hours after BV VB1-SpCas9-eGFP transduction, MC38 cells were collected, stained with anti-mouse PD-L1 antibody, washed with PBS and analyzed with flow cytometry to determine the eGFP expression in MC38 cells and PD-L1 protein on MC38 cell membrane (FIGS. 2A and 2B).

Fluorescent imaging (FIG. 2C) and flow cytometry analysis (FIGS. 2D and 2E) were used to assess the effect of MNP on in vitro transduction in different medium conditions. Adult mouse serum (AMS) was utilized to mimic complement factors present in in vivo conditions. In this regard, transduction of eGFP by BV eGFP, BV VB1-SpCas9-eGFP was measured in both F12 medium and F12 medium with 50% AMS was measured, while eGFP transduction via BV-MNP eGFP and BV-MNP VB1-SpCas9-eGFP was measured in F12 medium with 50% AMS was measured. Fluorescent imaging was employed to visual eGFP transduction via BV eGFP and BV-MNP eGFP (FIG. 2C). Flow cytometry analysis was performed 24 hours following cell transduction via BV eGFP and BV-MNP (FIG. 2D) and both 24 hours and 48 hours following cell transduction via BV VB1-SpCas9-eGFP and BV-MNP VB1-SpCas9-eGFP (FIG. 2E).

Flow cytometry analysis was used to assess immune checkpoint disruption facilitated by BV-mediated and BV-MNP-mediated transduction. In this regard, PD-L1 knockout in MC38 cells facilitated by BV VB1-SpCas9-eGFP and BV-MNP VB1-SpCas9-eGFP in F12 and/or F12 with 50% AMS was measured using flow cytometry analysis (FIG. 2F).

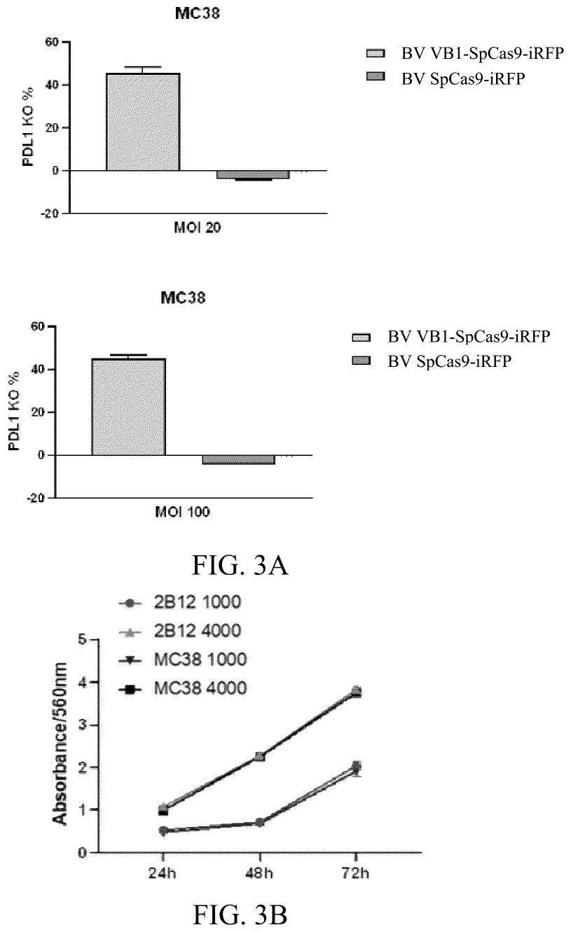

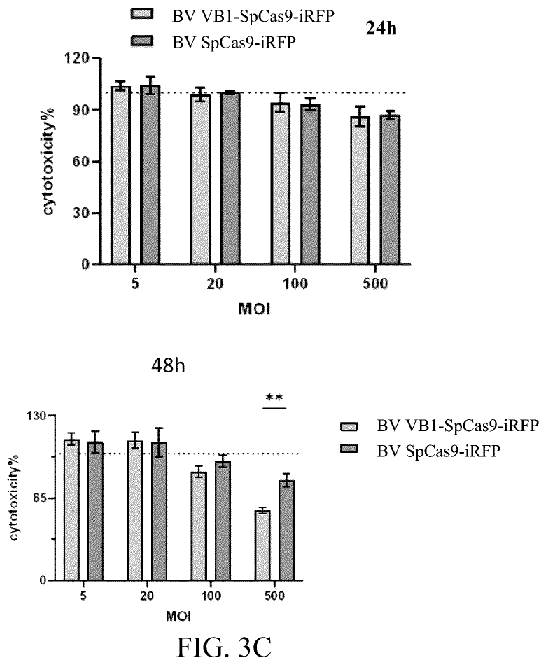

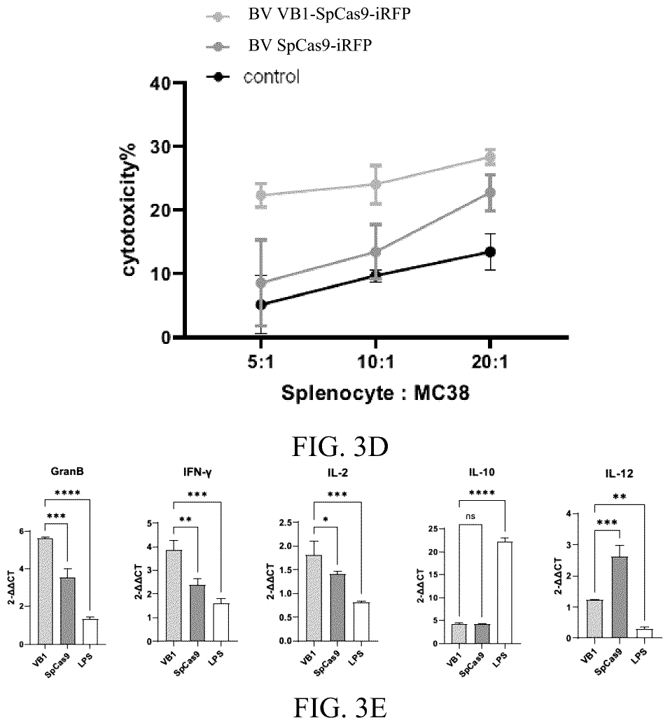

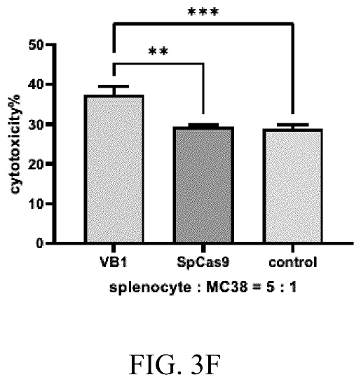

Flow cytometry was used to assess immune checkpoint disruption facilitated by BV-mediated transduction using gRNA VB1 versus SpCas9 alone. In this regard, PD-L1 knockout facilitated by BV VB1-SpCas9-iRFP and BV SpCas9-iRFP in MC38 cells at different MOIs was measured using flow cytometry analysis (FIG. 3A). 4×10⁴ MC38 cells were seeded in 24-well plate each well. 24 hours later, BV VB1-SpCas9-iRFP and BV SpCas9-iRFP were added into the fresh medium and cultured with MC38 cells, respectively. 48 hours after BV transfection, MC38 cells were collected, stained with anti-mouse PD-L1 antibody, washed with PBS, and analyzed with flow cytometry to determine PD-L1 protein on MC38 cell membrane. PD-L1 knockout (KO) percentage was calculated by control MC38 (almost 100%) subtracting treatment group. Cell growth between MC38 cells post BV VB1-SpCas9-iRFP and BV SpCas9-iRFP transduction was also assessed via CCK8 assay purchased from Dojindo laboratories (FIG. 3C). The growth between MC38 cells and 2B12 cells (a PD-L1 knockout clone selected from MC38 cells treated with pX330-VB1) using CCK8 assay (FIG. 3B).

To assess the extent to which an immune response was invoked in vitro as a result of BV-mediated transduction using gRNA VB1 versus SpCas9 alone, cytotoxicity (FIGS. 3D and 3F) and gene expression of select genes (IFN-γ, GranB, IL-2, and IL-10) (FIG. 3E) in MC38 cells transduced with BV VB1-SpCas9-iRFP and BV SpCas9-iRFP were incubated with splenocytes for 24 hours before analysis with cell counting kit 8 (CCK8) assay (for cytotoxicity) and RT-qPCR (for gene expression). With respect to cytotoxicity, splenocyte:MC38 cell ratios of 5:1, 10:1, and 20:1 were examined (FIG. 3D).

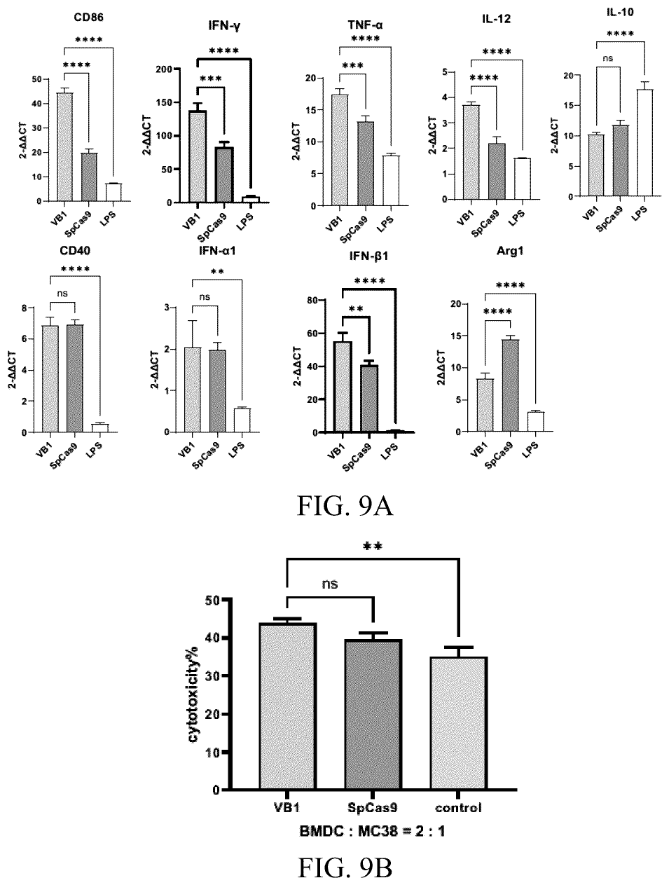

To further assess the extent to which an immune response was invoked in vitro as a result of BV-mediated transduction using gRNA VB1 versus SpCas9 alone, cytotoxicity (FIG. 9B) and gene expression of select genes (CD86, IFN-γ, TNF-α, IL-2, IL-10, CD40, IFN-α1, IFN-β1, and Arg1) (FIG. 9A) in MC38 cells transduced with BV VB1-SpCas9-iRFP and BV SpCas9-iRFP were incubated with bone marrow derived dendritic cells (BMDCs) for 24 before analysis with cell counting kit 8 (CCK8) assay (for cytotoxicity) and RT-qPCR (for gene expression). With respect to cytotoxicity, BMDC:MC38 cell ratios of 2:1 was examined (FIG. 9B).

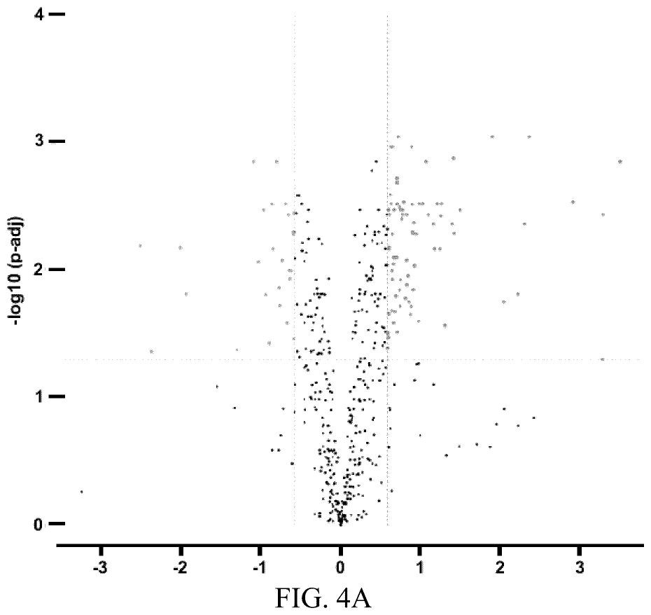

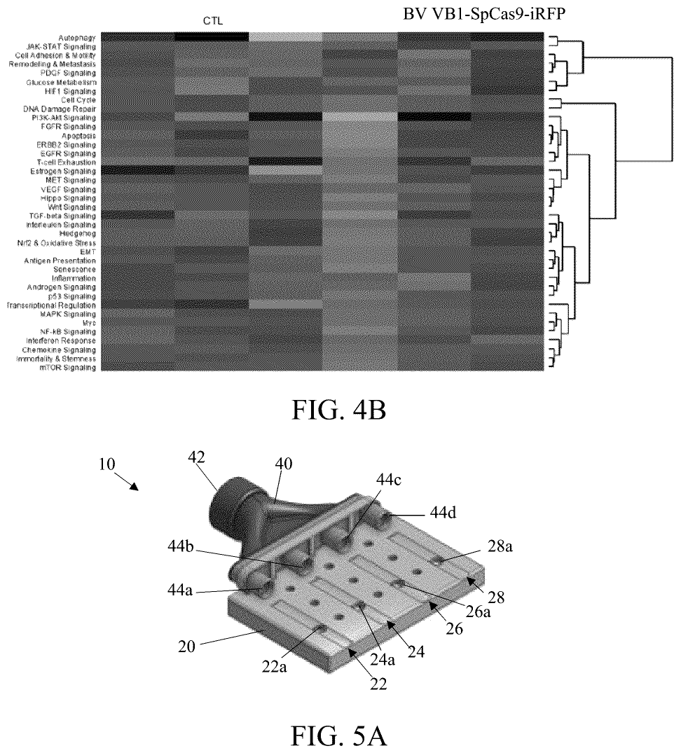

Gene expression differences in control MC38 cells versus MC38 cells transduced with BV VB1-SpCas9-iRFP was assessed via volcano map (FIG. 4A) and heat map (FIG. 4B). RNA was isolated from MC38 cells and BV-transfected MC38 cells and analyzed using the nCounter® tumor signaling 360 panel (NanoString) to investigate changes in signaling pathways.

### BV-Mediated Transduction In Vivo

C57BL6 mice, a colon cancer model, were utilized to asses BV-mediated transduction in vivo.

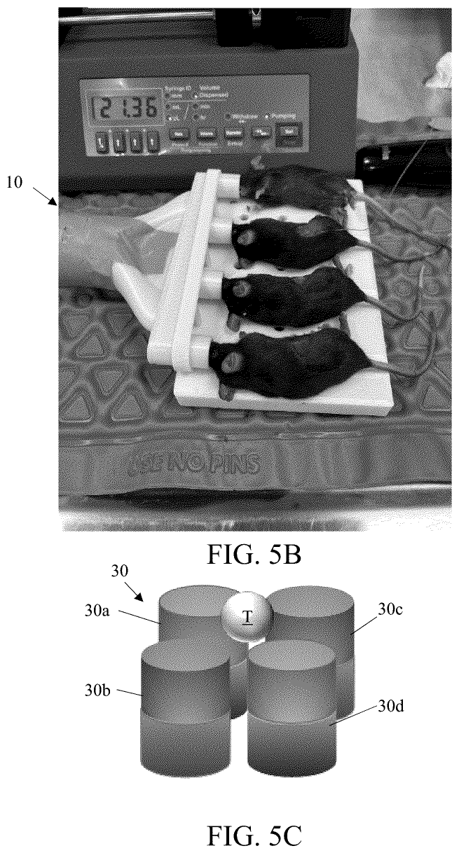

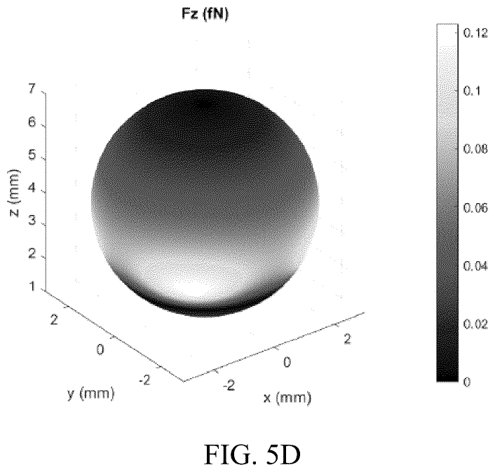

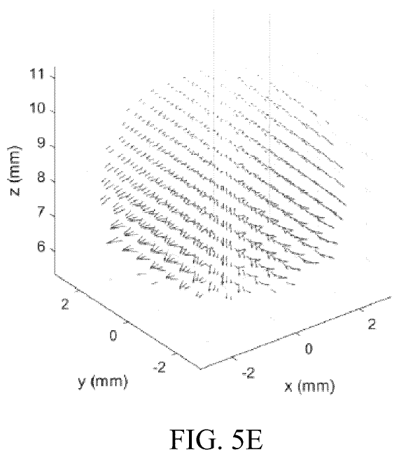

Six to eight week old C57BL6 mice were inoculated with 5×10⁵ MC38 cells subcutaneously on the right flank. When the tumor volume reached 50 mm³, the mice received intratumor infusions via a syringe pump every three days for a total of four times. Test group was administered BV-MNP VB1-SpCas9-iRFP, and control groups received PBS, MNP, BV VB1-SpCas9-iRFP, or αPD-L1 injection, where the αPD-L1 group received intraperitoneal (i.p.) injection. Following each infusion, BV-MNP VB1-SpCas9-iRFP mice were placed on a magnetic bed 10 developed in-house and configured to enhance the transduction of BV VB-SpCas9-iRFP in target locations within the mice, namely the tumor region. Referring now to FIGS. 5A-5C, the bed includes a platform 20 on which one or more subjects can be placed. Specifically, in this case, the platform 20 defines a total of four bays 22, 24, 26, 28, where each bay 22, 24, 26, 28 is configured to receive a C57BL6 mouse. Each bay 22, 24, 26, 28 includes a well 22a, 24a, 26a, 28a which is defined by a portion of the platform 20 corresponding to the bay 22, 24, 26, 28, and which is configured to, at least partially, receive the tumor of a C57BLC mouse. Each bay 22, 24, 26, 28 is provided with a magnetic device 30 configured to provide a magnetic field in an area corresponding to the well 22a, 24a, 26a, 28a. In this case, the magnetic device 30 of each bay 22, 24, 26, 28 was defined by four cylindrical neodymium iron boron (NdFeB) magnets 30a, 30b, 30c, 30d integrated into the platform 20 and arranged to provide a quadrupole magnet, such that, when the tumor, T, of a C57BL6 mouse is deposited in the well 22a, 24a, 26a, 28a of the bay 22, 24, 26, 28, the tumor, T, is centrally positioned relative to the four magnets 30a, 30b, 30c, 30d. In this case, the magnetic bed 10 also included a manifold 40 attached to the platform 20 and configured to supply a C57BLC with a flow of air containing oxygen and isoflurane while being treated on the magnetic bed 10. To this end, the manifold 40 an inlet 42 for receiving an inflow of air and an outlet 44a, 44b, 44c, 44d for delivering the flow of air to subjects in the respective bays 22, 24, 26, 28. In this regard, and in this case, the outlet 44a, 44b, 44c, 44d was actually defined by four outlets, with each outlet corresponding to one of the bays 22, 24, 26, 28. Thus, in this case, the manifold 40 defines, and thus can be characterized as including, a flow path with four branches. The platform 20 and manifold 40 were each constructed via three-dimensional (3D) printing using commercially available thermoplastics. In this case, the platform 20 and manifold 40 were integrally formed as a unitary component and the magnets 30a, 30b, 30c, 30d for each bay 22, 24, 26, 28 were deposited in openings defined by a bottom surface of the platform 20 (not shown).

During treatment on the magnetic bed 10, the tumor of each BV-MNP VB1-SpCas9-iRFP mouse was centered on top of a quadrupole of cylindrical neodymium iron boron (NdFeB) magnets 30a, 30b, 30c, 30d (FIGS. 5A-5C). Conformation of the quadrupole magnets and the location of the tumor were optimized through numerical simulations using a MATLAB program developed in-house (FIGS. 5D and 5E).

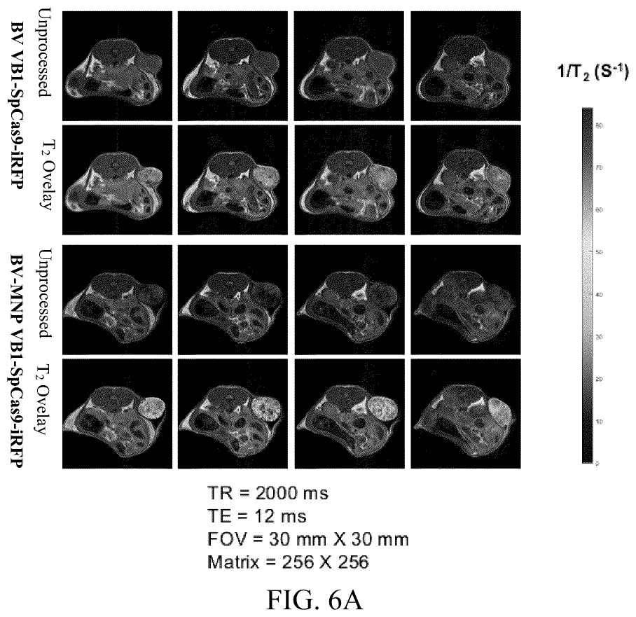

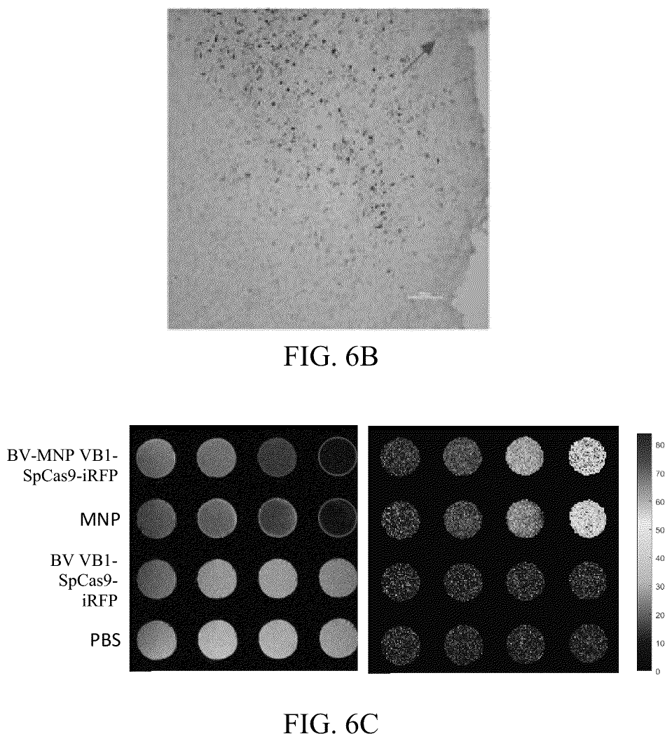

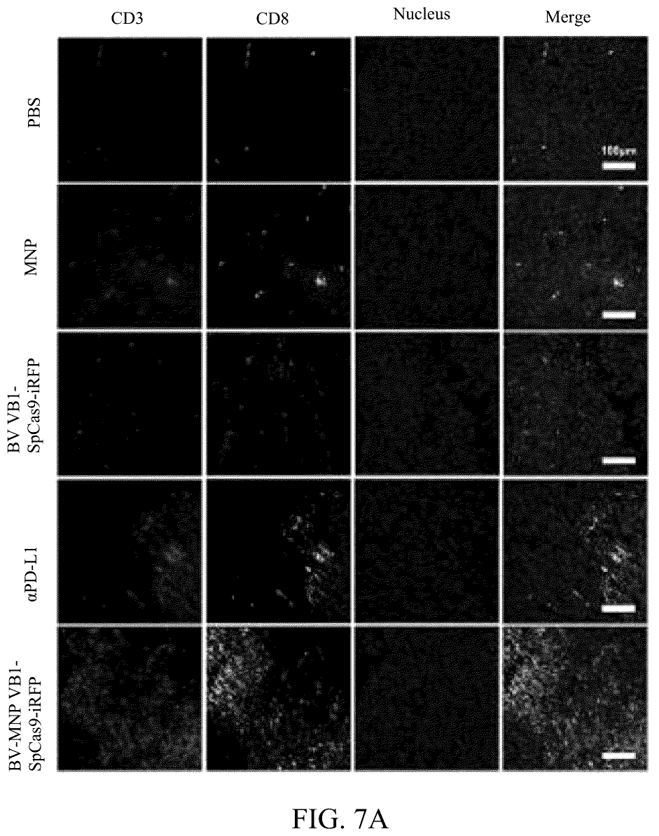

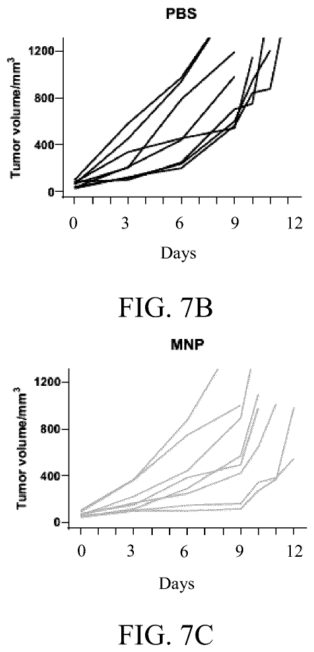

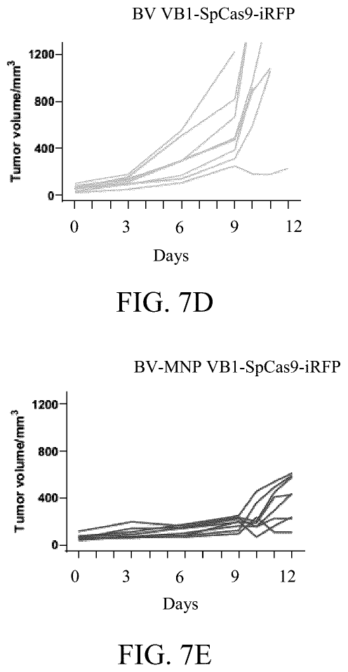

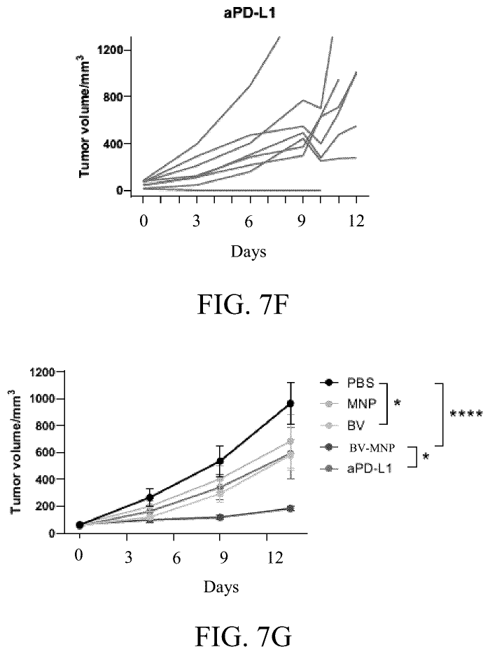

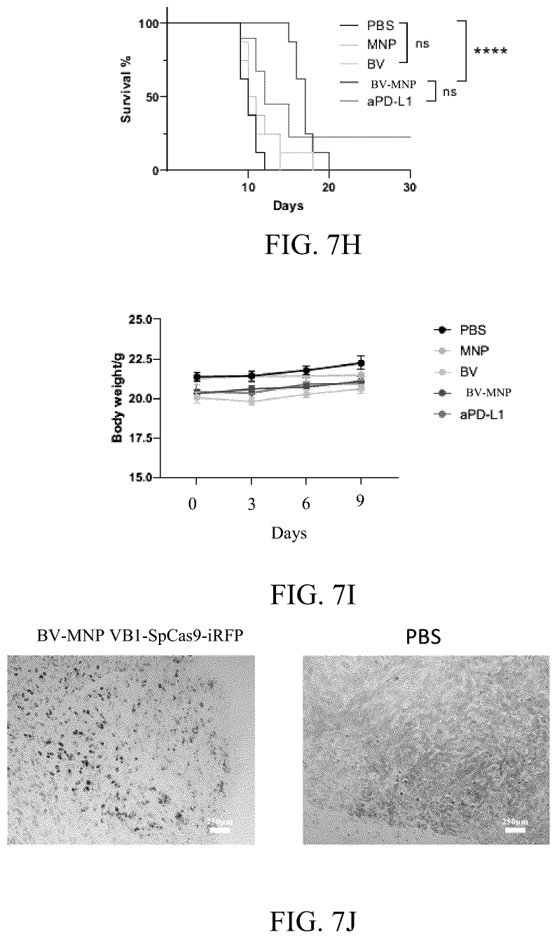

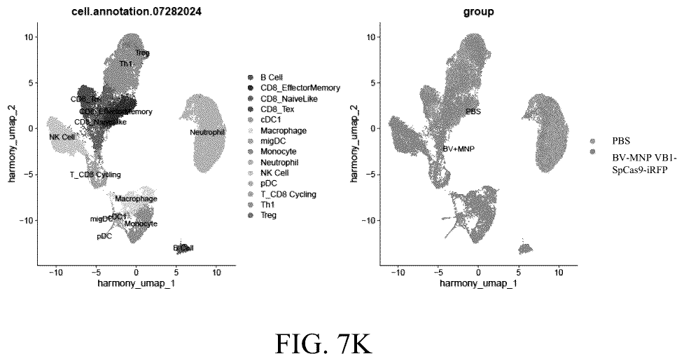

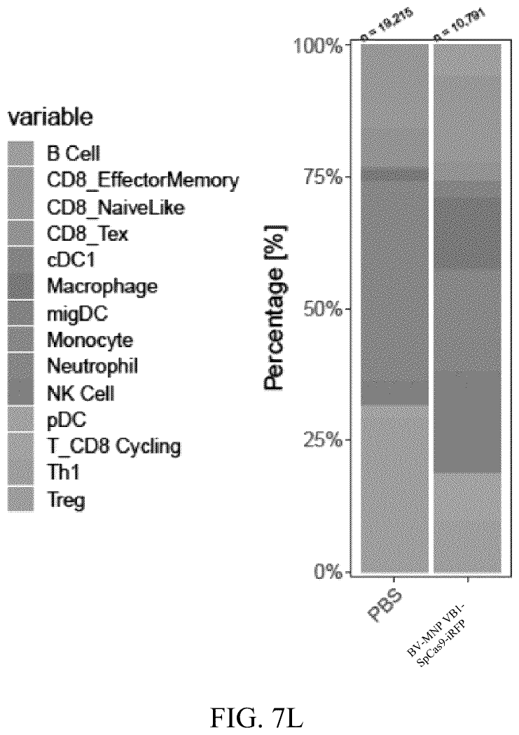

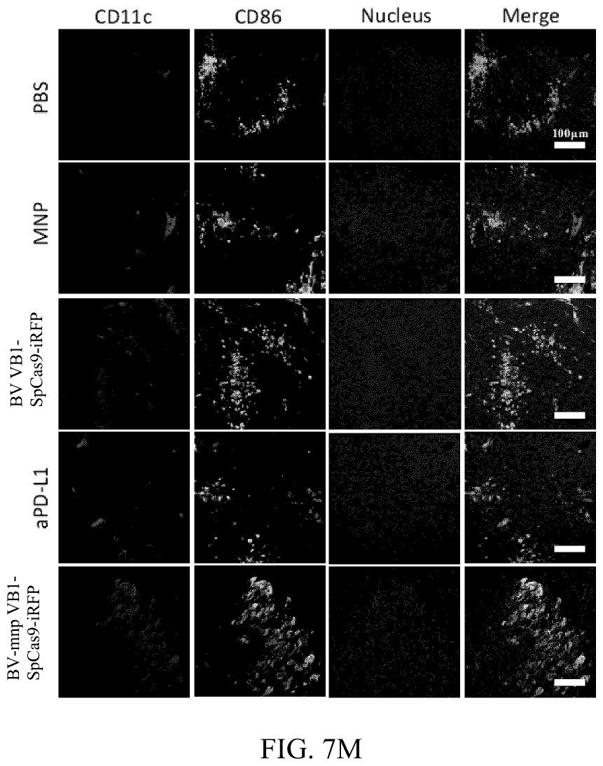

BV VB1-SpCas9-iRFP and BV-MNP VB1-SpCas9-iRFP mice were subjected to MRI imaging and T₂ mapping to assess whether MNPs and magnetic activation provide a viable means of tracking delivery of BV-mediated Cas nuclease and gRNA delivery to target cells (FIGS. 6A and 6C). A cryosection of tumors infused with BV-MNP VB1-SpCas9-iRFP was also taken to confirm MNP migration in response to application of the applied magnetic field (FIG. 6B). Prussian blue staining was also performed on BV-MNP VB1-SpCas9-iRFP and PBS groups to confirm presence of MNPs in tumor sections (FIG. 7J)

To assess whether BV-mediated delivery of VB1 and SpCas9 triggered an immune response, immunostaining of tumor sections was performed on each of the PBS, MNP, BV VB1-SpCa9-iRFP, αPD-L1 antibody, and BV-MNP VB1-Spcas9-iRFP groups. Staining was performed for CD3 and CD8 (FIG. 7A), CD11c and CD86 (FIG. 7M), and NK1.1 (not shown). To assess whether BV-mediated delivery of VBland SpCas9 suppressed tumor growth, tumor volume was measured in each of the PBS, MNP, BV VB1-SpCa9-iRFP, αPD-L1 antibody, and BV-MNP VB1-Spcas9-iRFP groups with vernier caliper and calculated with known formula (V=0.5×(L×W²)) (FIGS. 7B-7G). Survival percentage and body weight changes in each respective group was also measured (FIGS. 7H and 7I).

To assess BV-MNP VB1-SpCas9-iRFP transduction effect in various immune cellular components the tumors of C75BLC mice implanted with murine MC338 cancer cells, analysis was performed on immune cells isolated from BV-MNP VB1-SpCas9-iRFP and PBS treated tumors using Harmony^{26, 27} and Umap²⁸ programs (FIG. 7K). Data generated from such assessment was subsequently utilized to determine the percentage of various immune cellular components present in the tumors of C57BLC mice treated with PBS versus BV-MNP VB1-SpCas9-iRFP (FIG. 7L). BV-MNP VB1-SpCas9-iRFP treated tumors exhibited a significant increase in cycling CD8 T cells, NK cells, and dendritic cells.

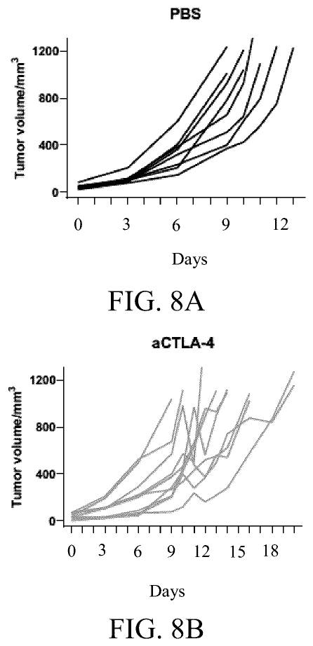

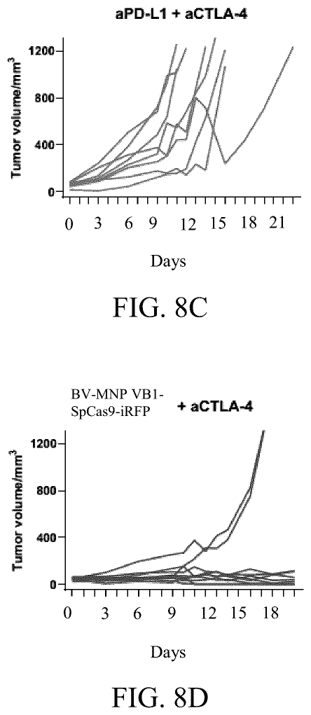

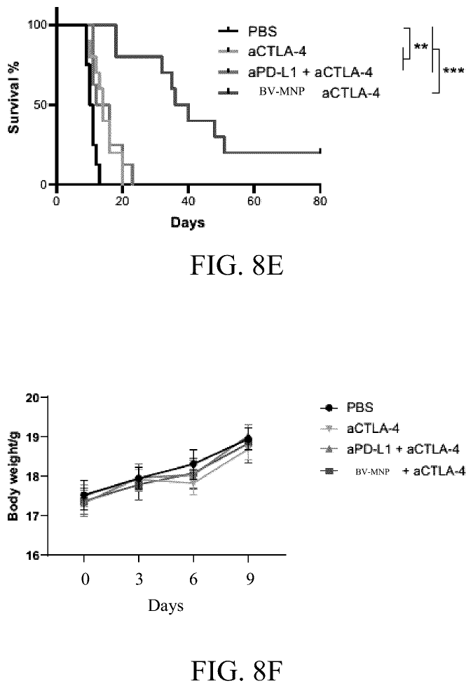

Subsequent studies were also conducted to determine whether the combined effects of BV-MNP VB1-SpCas9-iRFP and αCTLA-4, which targets complementary immune inhibitory pathway, could improve treatment outcomes in C57BLC mice implanted with murine MC38 cancer cells. In this regard, once tumor size reached 50 mm³, mice were divided into four groups, with each group receiving tumor injections of PBS, αCTLA-4, αPD-L1 and αCTLA-4, or BV-MNP VB1-SpCas9-iRFP and αCTLA-4 consistent with the techniques described above. Mice treated with BV-MNP VB1-SpCas9-iRFP were subjected to magnetic bed treatment consistent with that described above. Tumor volume, survival rate, and body weight were assessed in each of the groups (FIGS. 8A-8F).

### Results/Discussion

### BV-Mediated Transduction In Vitro

Effect of MNP amount on eGFP transduction. Increased amounts of MNP conjugated to BV was found to provide increased eGFP expression percentages in MC38 cells transduced by BV-MNP VB1-SpCas9-eGfp (FIG. 1D).

Duration of BV-mediated transduction. Transduction of eGFP in MC38 cells mediated by BV was found to be transitory, gradually decreasing over the course of a week post-transduction before eGFP was no longer expressed (FIG. 1E).

Effect of CRISPR system on eGFP expression and immune checkpoint inhibition. eGFP transduction in MC38 cells mediated by BV VB1-SpCas9-eGFP was found to significantly reduce the amount of PD-L1 protein on the cell membrane of MC38 cells while still facilitating good eGFP expression at different MOIs (FIGS. 2A and 2B).

Effect of complement immune system action on BV transduction. Consistent with previous findings, transduction mediated by BV alone was largely neutralized in conditions (F12 with 50% AMS) mimicking complement system action. However, the attachment of MNPs to the BV was found to significantly enhance transduction in such conditions (FIGS. 2C-2E). Compared with F12 group, the BV-MNP VB1-SpCas9-eGFP treatment did not achieve that high PD-L1 knockout efficiency, but when compared with F12+AMS group (mimicking complement system action), in which BV VB1-SpCas9-eGFP was used alone, a higher percentage of PD-L1 knockout was detected (FIG. 2F). Thus, as expected, it was found that AMS could inactivate BV and reduce desired gene editing outcome but that the combination of BV and MNP rescued part of the gene editing outcome.

PD-L1 knockout efficiency provided by gRNA. BV-mediated transduction utilizing guide RNA sequence VB1 was found to significantly improve PD-L1 knockout percentage in MC38 cells at different MOIs as evidenced by comparing BV VB1-SpCas9-iRFP and BV SpCas9-iRFP in FIG. 3A.

Effect of BV-mediated PD-L1 knockout on cell growth. 2B12 cells corresponding to PD-L1 knockout did not exhibit significantly different growth rate as compared to control MC38 cells, indicating that PD-L1 knockout facilitated by BV-mediated transduction may not adversely affect growth rate (FIG. 3B). A comparison of cell growth of MC38 cells transduced by BV VB1-SpCas9-iRFP versus cell growth of MC38 cells transduced by BV SpCas9-iRFP indicated that cell growth was not significantly affected at MOIs lower than 100 and after 24 h post-transduction (FIG. 3C).

Splenocyte cytotoxicity toward BV transduced cells. Splenocytes showed increased cytotoxicity toward MC38 cells transduced by BV VB1-SpCas9-iRFP relative to those transduced by BV SpCas9-iRFP and controls (FIGS. 3D and 3E), suggesting that BV alone induced immune response and that the response was enhanced by PD-L1 blockade. Splenocyte cytotoxicity toward MC38 cells was also found to increase at increased splenocyte to MC38 ratios (FIG. 3D).

Gene expression in splenocytes co-cultured with MC38 cells transduced by BV. When incubated with splenocytes, MC38 cells transduced with BV SpCas9-iRFP and BV VB1-SpCas9-iRFP were found to induce higher expression of GranB, IFN-γ, IL-2, and IL12 and lower expression of IL-10 compared to LPS treated cells, indicating that BV transfection could stimulate strong anti-tumor immune response (FIG. 3E). In assessing the pathway expression impacted by BV VB1-SpCas9 transduction, 794 genes were detected in total, including 6 positive genes, 8 negative genes, 20 housekeeping genes, and 760 genes from 48 signaling pathways. 506 genes were analyzed, with 26 genes found to be significantly downregulated (p-Adj <0.05, fold change ≤−1.5), and 81 genes found to be significantly upregulated (p-Adj <0.05, fold change ≥1.5). BV VB1-SpCas transduction upregulated the expression of 31 signaling pathways and downregulated the expression of 7 signaling pathways in MC38 cells (FIGS. 4A and 4B). The signaling pathway analysis of gene expression in MC38 cells, with or without BV VB1-SpCas9 transduction, systematically examined the impact of BV VB1-SpCas9 transfection on these cells. Disruption of the PD-L1 gene by BV VB1-SpCas9 in MC38 cells activated the DNA damage repair pathway and PI3K-Akt pathways, and even lead to cell apoptosis. Furthermore, BV VB1-SpCas9 transfection triggered an immune response against viral infection in cells, activating pathways such as PI3K-Akt, TGF-β, interleukin, inflammation, MAPK, and NF-κB. Notably, the antigen presentation pathway was also activated, which enhances immune cell recognition and destruction of tumor cells.

BMDC cytotoxicity toward BV transduced cells. BMDCs showed increased cytotoxicity toward MC38 cells transduced by BV VB1-SpCas9-iRFP and BV SpCas9-iRFP relative to controls (FIG. 9B). No significant difference was observed with respect to cytotoxicity between MC38 cells transduced by BV VB1-SpCas9-iRFP and MC38 cells transduced by BV SpCas9-iRFP, suggesting that BV alone induced immune response.

Gene expression in BMDCs co-cultured with MC38 cells transduced by BV. When incubated with BMDC, MC38 cells transduced with BV SpCas9-iRFP and BV VB1-SpCas9-iRFP were found to induce higher expression of CD86, IFN-γ, TNF-α, IL-12, CD40, IFN-α1, IFN-γ1, and ARg1 and lower expression of IL-10 compared to LPS treated cells (FIG. 9A). BV VB1-SpCas9 transfection in MC38 cells could stimulate strong anti-tumor immune response from BMDC, which plays an important role in tumor immunotherapy.

### BV-Mediated Transduction In Vivo

### Intratumoral Infusion, Magnetic Activation, and Tracking of BV-MNP.

The high T₂ relaxivity of MNPs in BV-MNP VB1-SpCas9 was found to provide sufficient T₂ contrast to facilitate imaging of MNP with MRI (FIG. 6C). Accordingly, the conjugation of MNP to BV in BV-MNP carrying VB1-SpCas9-iRFP permitted in vivo tracking of the MNPs, and thus BV-MNP carrying VB1-SpCas9-iRFP, in the tumor of C57BLC infused with BV-MNP VB1-SpCas9-iRFP using MRI T₂ imaging. Tumors infused with BV VB1-SpCas9 (i.e., without MNPs conjugated to BV) were not trackable in vivo (FIG. 6A). Microscopic examination of tumor cryosection showed the distribution of MNP clusters near the needle track and the tumor peripheral in proximity to the magnets of the magnetic bed, indicating that the magnetic force applied by the magnetic bed was effective to direct BV-MNP VB1-SpCas9-iRFP as intended (FIG. 6B). Presence of MNPs in cells of BV-MNP VB1-SpCas9-iRfP group was confirmed with Prussian blue staining (FIG. 7I).

BV-MNP with VB1 gRNA increases the infiltration of CD3⁺ and CD8⁺ T cells, suppresses tumor growth, and increases survival in colon cancer model.

Immunostaining revealed a decrease of PD-L1 in tumor treated with BV-MNP VB1-SpCas9-iRFP. A moderate increase in CD3⁺ and CD8⁺ T cell infiltration in the tumors treated with MNP, BV VB1-SpCas9-iRFP, or αPD-L1 was observed, while a more pronounced increase in CD3⁺ and CD8⁺ T cell infiltration was observed in tumors treated with BV-MNP VB1-SpCas9-iRFP (FIG. 7A). The BV-MNP VB1-SpCas9-iRFP group also showed the most dendric cells (CD86⁺ and CD11c⁺) (FIG. 7M). There was no significant change, however, in T_{reg} among all groups (i.e., PBS, MNP, BV VB1-SpCas9-iRFP, αPD-L1, or BV-MNP VB1-SpCas9-iRFP). Compared to PBS, MNP alone, BV VB1-SpCas9-iRFP, and αPD-L1, BV-MNP VB1-SpCas9-iRFP significantly inhibited tumor growth (FIGS. 7B-7G). Compared to PBS and MNP alone, BV VB1-SpCas9-iRFP and BV-MNP VB1-SpCas9-iRFP improved survival rate. But αPD-L1 had a complete tumor remission (FIG. 7H). No significant difference in body weight was observed amongst groups (FIG. 7I).

Combination of αCTLA-4 and BV-MNP with VB1 gRNA suppresses tumor growth and increases survival in colon cancer model.

Since BV-MNP VB1-SpCas9-iRfP was found to suppress tumor progression, the combination of BV-MNP VB1-SpCas9-iRFP and αCTLA-4, which targets complementary immune inhibitory pathway, was examined as therapeutic. Surprisingly, C57BL6 mice treated with BV-MNP VB1-SpCas9-iRFP in combination with αCTLA-4 were found to exhibit significantly longer survival compared to groups treated with PBS, αCTLA-4 alone, and αPD-L1 and αCTLA-4. The median survival of the αPD-L1 and αCTLA-4 group was 14 days, while the BV-MNP VB1-SpCas9-iRFP and αCTLA-4 group was 38 days (FIG. 8E). Tumor volume was also generally found to be more significantly suppressed in the BV-MNP VB1-SpCas9-iRFP and αCTLA-4 group as compared to the other groups (FIGS. 8A-8D). There was no significant difference in the body weight among all groups (FIG. 8F).

### Conclusion

Taken together, the studies disclosed herein prove that BV-MNP VB1-SpCas9 disrupt mouse PD-L1 in tumor tissue and induce a robust immune response including significant infiltration of CD3⁺ and CD8⁺ T cells and dendritic cells. Additionally, intratumoral infusion of BV-MNP VB1-SpCas9 and systemic administration of an immune checkpoint inhibitor exhibits synergistic effects in tumor suppression. Furthermore, the studies demonstrate the use of MNPs in conjunction with BV and MRI imaging provides an effective means of tracking CRISPR system delivery in vivo.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Doudna J A. The promise and challenge of therapeutic genome editing. Nature. 2020; 578(7794):229-36. Epub 20200212. doi: 10.1038/s41586-020-1978-5. PubMed PMID: 32051598; PMCID: PMC8992613.
2. Maeder M L, Stefanidakis M, Wilson C J, Baral R, Barrera L A, Bounoutas G S, Bumcrot D, Chao H, Ciulla D M, DaSilva J A, Dass A, Dhanapal V, Fennell T J, Friedland A E, Giannoukos G, Gloskowski S W, Glucksmann A, Gotta G M, Jayaram H, Haskett S J, Hopkins B, Horng J E, Joshi S, Marco E, Mepani R, Reyon D, Ta T, Tabbaa D G, Samuelsson S J, Shen S, Skor M N, Stetkiewicz P, Wang T, Yudkoff C, Myer V E, Albright C F, Jiang H. Development of a gene-editing approach to restore vision loss in Leber congenital amaurosis type 10. Nat Med. 2019; 25(2):229-33. Epub 20190121. doi: 10.1038/s41591-018-0327-9. PubMed PMID: 30664785.
3. Cong L, Ran F A, Cox D, Lin S L, Barretto R, Habib N, Hsu P D, Wu X B, Jiang W Y, Marraffini L A, Zhang F. Multiplex Genome Engineering Using CRISPR/Cas Systems. Science. 2013; 339(6121):819-23. doi: 10.1126/science.1231143. PubMed PMID: ISI:000314874400049.
4. Canver M C, Smith E C, Sher F, Pinello L, Sanjana N E, Shalem O, Chen D D, Schupp P G, Vinjamur D S, Garcia S P, Luc S, Kurita R, Nakamura Y, Fujiwara Y, Maeda T, Yuan G C, Zhang F, Orkin S H, Bauer D E. BCL11A enhancer dissection by Cas9-mediated in situ saturating mutagenesis. Nature. 2015; 527(7577):192-7. Epub 20150916. doi: 10.1038/naturel5521. PubMed PMID: 26375006; PMCID: PMC4644101.
5. Lin Y N, Cradick T J, Brown M T, Deshmukh H, Ranjan P, Sarode N, Wile B M, Vertino P M, Stewart F J, Bao G. CRISPR/Cas9 systems have off-target activity with insertions or deletions between target DNA and guide RNA sequences. Nucleic Acids Res. 2014; 42(11):7473-85. doi: 10.1093/nar/gku402. PubMed PMID: ISI:000338769400063.
6. Tong S, Moyo B, Lee C M, Leong K, Bao G. Engineered materials for in vivo delivery of genome-editing machinery. Nat Rev Mater. 2019; 4(11):726-37. Epub 20191004. doi: 10.1038/s41578-019-0145-9. PubMed PMID: 34094589; PMCID: PMC8174554.
7. Wang Y, Hu J K, Krol A, Li Y P, Li C Y, Yuan F. Systemic dissemination of viral vectors during intratumoral injection. Mol Cancer Ther. 2003; 2(11):1233-42. Epub 2003/11/18. PubMed PMID: 14617797.
8. Zhu H, Zhang L, Tong S, Lee C M, Deshmukh H, Bao G. Spatial control of in vivo CRISPR-Cas9 genome editing via nanomagnets. Nat Biomed Eng. 2019; 3(2):126-36. Epub 20181112. doi: 10.1038/s41551-018-0318¬7. PubMed PMID: 30944431; PMCID: PMC6450418.
9. Tong S, Zhu H, Bao G. Magnetic Iron Oxide Nanoparticles for Disease Detection and Therapy. Mater Today. 2019; 31:86-99. Epub 20190622. doi: 10.1016/j.mattod.2019.06.003. PubMed PMID: 32831620; PMCID: PMC7441585.
10. Ono C, Okamoto T, Abe T, Matsuura Y. Baculovirus as a Tool for Gene Delivery and Gene Therapy. Viruses. 2018; 10(9):510. Epub 20180919. doi: 10.3390/v10090510. PubMed PMID: 30235841; PMCID: PMC6164903.
11. Wang S, Balasundaram G. Potential cancer gene therapy by baculoviral transduction. Current gene therapy. 2010; 10(3):214-25. Epub 2010/04/21. PubMed PMID: 20402653.
12. Wilson M W, Kerlan R K, Jr., Fidelman N A, Venook A P, LaBerge J M, Koda J, Gordon R L. Hepatocellular carcinoma: regional therapy with a magnetic targeted carrier bound to doxorubicin in a dual M R imaging/conventional angiography suite-initial experience with four patients. Radiology. 2004; 230(1):287-93. doi: 10.1148/radiol.2301021493. PubMed PMID: 14695402.
13. Boyce F M, Bucher N L. Baculovirus-mediated gene transfer into mammalian cells. Proc Natl Acad Sci USA. 1996; 93(6):2348-52. PubMed PMID: 8637876; PMCID: PMC39799.
14. Mansouri M, Bellon-Echeverria I, Rizk A, Ehsaei Z, Cianciolo Cosentino C, Silva C S, Xie Y, Boyce F M, Davis M W, Neuhauss S C, Taylor V, Ballmer-Hofer K, Berger I, Berger P. Highly efficient baculovirus-mediated multigene delivery in primary cells. Nat Commun. 2016; 7:11529. Epub 2016/05/05. doi: 10.1038/ncomms11529. PubMed PMID: 27143231; PMCID: PMC4857464.
15. Kost T A, Condreay J P, Jarvis D L. Baculovirus as versatile vectors for protein expression in insect and mammalian cells. Nat Biotechnol. 2005; 23(5):567-75. Epub 2005/05/07. doi: 10.1038/nbt1095. PubMed PMID: 15877075; PMCID: PMC3610534.
16. Sung L Y, Chen C L, Lin S Y, Li K C, Yeh C L, Chen G Y, Lin C Y, Hu Y C. Efficient gene delivery into cell lines and stem cells using baculovirus. Nat Protoc. 2014; 9(8):1882-99. Epub 20140710. doi: 10.1038/nprot.2014.130. PubMed PMID: 25010908.
17. Fay N, Pante N. Nuclear entry of DNA viruses. Front Microbiol. 2015; 6:467. Epub 20150513. doi: 10.3389/fmicb.2015.00467. PubMed PMID: 26029198; PMCID: PMC4429625.
18. Wu C, Lin J, Hong M, Choudhury Y, Balani P, Leung D, Dang L H, Zhao Y, Zeng J, Wang S. Combinatorial control of suicide gene expression by tissue-specific promoter and microRNA regulation for cancer therapy. Mol Ther. 2009; 17(12):2058-66. Epub 20091006. doi: 10.1038/mt.2009.225. PubMed PMID: 19809402; PMCID: PMC2814389.
19. Welsh R M, Bahl K, Marshall H D, Urban S L. Type 1 Interferons and Antiviral CD8 T-Cell Responses. PLoS Pathogens. 2012; 8(1):e1002352. doi: 10.1371/journal.ppat.1002352.
20. Fuertes M B, Kacha A K, Kline J, Woo S-R, Kranz D M, Murphy K M, Gajewski T F. Host type I IFN signals are required for antitumor CD8+ T cell responses through CD8α+ dendritic cells. Journal of Experimental Medicine. 2011; 208(10):2005-16. doi: 10.1084/jem.20101159.
21. Airenne K J, Hu Y C, Kost T A, Smith R H, Kotin R M, Ono C, Matsuura Y, Wang S, Yla-Herttuala S. Baculovirus: an insect-derived vector for diverse gene transfer applications. Mol Ther. 2013; 21(4):739-49. Epub 2013/02/27. doi: 10.1038/mt.2012.286. PubMed PMID: 23439502; PMCID: PMC3616530.
22. Chen C Y, Lin C Y, Chen G Y, Hu Y C. Baculovirus as a gene delivery vector: recent understandings of molecular alterations in transduced cells and latest applications. Biotechnol Adv. 2011; 29(6):618-31. Epub 2011/05/10. doi: 10.1016/j.biotechadv.2011.04.004. PubMed PMID: 21550393.
23. Hofmann C, Strauss M. Baculovirus-mediated gene transfer in the presence of human serum or blood facilitated by inhibition of the complement system. Gene Ther. 1998; 5(4):531-6. Epub 1998/06/06. doi: 10.1038/sj.gt.3300607. PubMed PMID: 9614578.
24. Michlits G, Jude J, Hinterndorfer M, de Almeida M, Vainorius G, Hubmann M, Neumann T, Schleiffer A, Burkard T R, Fellner M, Gijsbertsen M, Traunbauer A, Zuber J, Elling U. Multilayered VBC score predicts sgRNAs that efficiently generate loss-of-function alleles. Nature methods. 2020; 17(7):708-16. Epub 20200608. doi: 10.1038/s41592-020-0850-8. PubMed PMID: 32514112.
25. U.S. Pat. No. 10,286,073.
26. BroadInstitute.org, “Harmony” available at https://portals.broadinstitute.org/harmony/(last visited Nov. 6, 2024).
27. GitHub, “immunogenomics/harmony” released Feb. 19, 2020, available at https://github.com/immunogenomics/harmony (last visited Nov. 6, 2024).
28. Github, “Imcinnes/umap” initial release Jun. 4, 2020, available at https://github.com/lmcinnes/umap/releases (last visited Nov. 6, 2024).
29. Sun, S. et al. Monodisperse MFe204 (M=Fe, Co, Mn) nanoparticles. J. Am. Chem. Soc. 126, 273-279 (2004).
30. Tong, S, Hou, S., Ren, B., Zheng, Z. & Bao, G. Self-assembly of phospholipid-PEG coating on nanoparticles through dual solvent exchange. Nano let 11, 3720-3726 (2011).

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for targeted in vivo gene editing, comprising:
packaging a clustered regularly interspaced palindromic repeat (CRISPR) associated protein 9 (Cas9) and a guide RNA into a baculovirus vector (BV), the guide RNA having homology to an immune checkpoint gene impeding an immune response in a subject;
attaching a plurality of magnetic nanoparticles (MNPs) to the BV to form a BV-MNP complex;
administering the BV-MNP complex carrying the Cas9 and the guide RNA to the subject to thereby inhibit the immune checkpoint gene.

2. The method of claim 1, wherein inhibition of the immune checkpoint gene is characterized by at least one of a decrease in expression or activity of a protein encoded by the immune checkpoint gene and a decrease in tumor volume in the subject.

3. The method of claim 2, wherein the immune checkpoint gene is programmed death-ligand 1 (PD-L1).

4. The method of claim 1, wherein the immune response impeded by the immune checkpoint gene is increased infiltration of at least one of lymphocytes and dendritic cells into a tissue of the subject, and wherein inhibition of the immune checkpoint gene promotes the immune response in the subject.

5. The method of claim 4, wherein the tissue of the subject is cancerous tumor tissue.

6. The method of claim 5, wherein the cancer is colon cancer.

7. The method of claim 4, wherein inhibition of the immune checkpoint gene results in increased infiltration of lymphocytes including CD3 lymphocytes, CD8 lymphocytes, or a combination thereof into the tissue of the subject.

8. The method of claim 1, wherein an immune checkpoint inhibitor is administered to the subject in combination with the BV-MNP complex carrying the Cas9 and the guide RNA.

9. The method of claim 8, wherein the immune checkpoint inhibitor is alpha cytotoxic T-lymphocyte associated protein 4 (αCTLA-4).

10. The method of claim 1, and further comprising a step of:
applying a magnetic field to a target tissue in the subject subsequent to administration of the BV-MNP complex carrying the Cas9 and the guide RNA, the target tissue corresponding to an area of the subject where the immune response is desired.

11. The method of claim 1, wherein the guide RNA comprises the sequence of SEQ ID NO: 1, SEQ ID NO: 2, SEQ ID NO: 3, SEQ ID NO: 4, SEQ ID NO: 5, or SEQ ID: NO: 6.

12. A method for promoting an immune response in a subject, comprising:
administering a baculovirus vector (BV)-magnetic nanoparticle (MNP) (BV-MNP) complex carrying a clustered regularly interspaced palindromic repeat (CRISPR) associated protein 9 (Cas9) and a guide RNA to the subject, the guide RNA having homology to an immune checkpoint gene impeding the immune response in the subject;
wherein administration of the BV-MNP complex carrying the Cas9 and the guide RNA inhibits the immune checkpoint gene.

13. The method of claim 12, wherein the immune checkpoint gene is programmed death-ligand 1 (PD-L1).

14. The method of claim 13, wherein the guide RNA comprises the sequence of SEQ ID NO: 1, SEQ ID NO: 2, SEQ ID NO: 3, SEQ ID NO: 4, SEQ ID NO: 5, or SEQ ID: NO: 6.

15. The method of claim 12, wherein the immune response impeded by the immune checkpoint gene is increased infiltration of at least one of lymphocytes and dendritic cells into cancerous tissue of the subject.

16. The method of claim 15, wherein the BV-MNP complex carrying the Cas9 and the guide RNA inhibits the immune checkpoint gene is administered in the cancerous tissue, and wherein the method further comprises a step of:
applying a magnetic field to the cancerous tissue subsequent to administration of the BV-MNP carrying the Cas9 and the guide RNA.

17. The method of claim 12, wherein alpha cytotoxic T-lymphocyte associated protein 4 (αCTLA-4) is administered in combination with the BV-MNP complex carrying the Cas9 and the guide RNA.

18. A method for detecting in vivo delivery of a clustered regularly interspaced short palindromic repeat (CRISPR) system, comprising:
administering a baculovirus vector (BV)-magnetic nanoparticle (MNP) (BV-MNP) complex carrying a CRISPR-associated protein 9 (Cas9) and a guide RNA to a subject, the guide RNA having homology to a gene of the subject;
imaging the subject or a biopsy acquired from the subject with magnetic resonance imaging (MRI) to acquire one or more images; and
detecting the presence or absence of the BV-MNP complex based on the presence or absence of a depiction of MNPs in the one or more images.

19. The method of claim 18, and further comprising a step of:
applying a magnetic field to a target tissue of the subject subsequent to administration of the BV-MNP carrying the Cas9 and the guide RNA and prior to imaging of the subject or the biopsy acquired from the subject.

20. A method for inhibiting tumor growth in a subject, comprising:
administering a baculovirus vector (BV)-magnetic nanoparticle (MNP) (BV-MNP) complex carrying a clustered regularly interspaced palindromic repeat (CRISPR) associated protein 9 (Cas9) and a guide RNA to tumor tissue in the subject, the guide RNA having homology to an immune checkpoint gene impeding infiltration of at least one of lymphocytes and dendritic cells into the tumor tissue;
wherein administration of the BV-MNP complex carrying the Cas9 and the guide RNA inhibits the immune checkpoint gene.

21. The method of claim 20, wherein the immune checkpoint gene is programmed death-ligand 1 (PD-L1).

22. The method of claim 20, wherein alpha cytotoxic T-lymphocyte associated protein 4 (αCTLA-4) is administered in combination with the BV-MNP complex carrying the Cas9 and the guide RNA.
